(19) United States
(12) Patent Application Publication
(10) Pub. No.: US 2026/0070995 A1
(43) Pub. Date: Mar. 12, 2026

(54) METHODS FOR TREATING BULLOUS PEMPHIGOID BY ADMINISTERING AN IL-4R ANTAGONIST

(71) Applicants: Marcella RUDDY, Wellesley, MA (US); Regeneron Pharmaceuticals, Inc., Tarrytown, NY (US); Sanofi Biotechnology, Gentilly (FR)

(72) Inventors: Deepti R. DESHPANDE, Short Hills, NJ (US); Elizabeth LAWS, Gentilly (FR); Leda MANNENT, Gentilly (FR); Marcella RUDDY, Wellesley, MA (US); Arsalan Q. SHABBIR, Mt. Kisco, NY (US)

(21) Appl. No.: 19/323,832

(22) Filed: Sep. 9, 2025

Related U.S. Application Data
(60) Provisional application No. 63/699,015, filed on Sep. 25, 2024.
(60) Provisional application No. 63/754,417, filed on Feb. 5, 2025.
(60) Provisional application No. 63/767,752, filed on Mar. 6, 2025.
(60) Provisional application No. 63/798,159, filed on May 1, 2025.
(60) Provisional application No. 63/826,166, filed on Jun. 18, 2025.

(30) Foreign Application Priority Data
Sep. 9, 2024 (EU) 24306470.6

(51) Int. Cl. C07K 16/28; A61K 39/00; A61K 45/06; A61P 17/00; A61P 37/02

(52) U.S. Cl. CPC C07K 16/2866; A61K 45/06; A61P 17/00; A61P 37/02; A61K 2039/505; A61K 2039/545

(57) ABSTRACT

Methods for treating bullous pemphigoid in a subject are provided. In one aspect, the methods comprise administering to a subject having bullous pemphigoid one or more doses of an interleukin-4 receptor (IL-4R) antagonist, such as an anti-IL-4R antibody or antigen-binding fragment thereof.

### CROSS-REFERENCE TO RELATED APPLICATIONS

This application claims priority to European Patent Application No. 24306470.6, filed Sep. 9, 2024, and to United States Provisional Patent Application Nos. 63/699,015, filed Sep. 25, 2024; 63/754,417, filed Feb. 5, 2025; 63/767,752, filed Mar. 6, 2025; 63/798,159, filed May 1, 2025; and 63/826,166, filed Jun. 18, 2025; the entire contents of each of which are incorporated by reference herein.

### REFERENCE TO A SEQUENCE LISTING

This application incorporates by reference a computer readable Sequence Listing in ST.26 XML format, titled 11894US01_Sequence, created on Sep. 4, 2025 and containing 354,217 bytes.

### FIELD OF THE INVENTION

The present disclosure relates to the use of interleukin-4 receptor (IL-4R) antagonists for the treatment of bullous pemphigoid.

### BACKGROUND

Bullous pemphigoid (BP) is a rare, autoimmune subepidermal blistering disease with a clinical course that may last from months to years. The disease characteristically presents in the elderly with a prodromal, non-bullous phase which may progress over weeks to years to a generalized, pruritic bullous eruption. In the bullous phase, BP is characterized by large, tense, serous, or hemorrhagic bullae arising on erythematous, urticarial, or eczematous skin. Because of the intractable pruritus and the presence of bullous, eroded or impetiginized lesions, the disease often has a profound, negative impact on the quality of life (Kouris, et al., An Bras Dermatol 2016, 91:601-603). The estimated mortality rate during the first year in the bullous phase of disease varies between 10% and 40% (Bystryn et al., J Invest Dermatol 2005, 124(3):xx-xxi; Joly, et al., Arch Dermatol 2005, 141:691-698).

Clinical findings and histological features suggest that a type 2 or T-helper type 2 (Th2) cell-mediated immune response plays a central role in the pathogenesis of BP. In support of the type 2-mediated response, elevated blood eosinophils, increased total serum immunoglobulin E (IgE) concentrations, and elevated serum and blister-fluid levels of different soluble, inflammatory Th2 cell-response mediators have been described in large cohorts of patients with BP (Cozzani, et al., Eur J Dermatol 2018, 28:396-397).

While high doses of oral corticosteroids (OCS) (e.g., doses of prednisone up to 0.75 mg/kg/day) have been considered the mainstay of treatment for many years, it has been clearly demonstrated that the prolonged use of high OCS doses are deleterious and directly responsible for a high rate of treatment side effects and contribute to the high mortality rate associated with this disease (see, e.g., Joly, et al., Arch Dermatol 2005, 141:691-698). Due to the toxicity of OCS in this elderly population, it is recommended that the cumulative dose of OCS be limited, and the high doses of OCS tapered expeditiously to the minimal effective dose of OCS that can control the disease (Venning, et al., Br J Dermatol 2012, 167:1200-1214). Moreover, to compensate for the high disease relapse rate (up to 80%) which occurs during OCS tapering, OCS-sparing immunosuppressive agents (such as mycophenolate mofetil, methotrexate, or rituximab) are frequently added. However, these treatments are known to have additional undesirable side effects. Highly potent topical corticosteroids (e.g., clobetasol propionate 0.05% cream) have been demonstrated to be effective for BP and may be safer than high doses of OCS (Joly, et al., J Invest Dermatol 2009, 129:1681-1687). However, despite their efficacy, highly potent topical corticosteroids can result in cutaneous atrophy, striae, and folliculitis. Long-term application of highly potent topical corticosteroids is also not recommended for sensitive skin areas or for application over a large body surface area, which may result in substantial systemic absorption with a resultant side effect profile similar to that of systemic corticosteroids. Therefore, there exists a significant unmet medical need for an effective treatment that has a favorable safety profile for long-term use for the treatment of BP.

### SUMMARY

In one aspect, methods for achieving sustained disease remission in the absence of oral corticosteroid therapy in a subject having bullous pemphigoid are provided. In some embodiments, the method comprises:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject to be treated has (i) a baseline Bullous Pemphigoid Disease Area Index (BPDAI) activity score ≥24, and (ii) a baseline pruritus Numerical Rating Score (NRS) 4.

In some embodiments, the subject to be treated is on a background therapy comprising an oral corticosteroid.

In another aspect, methods for reducing or eliminating the need for oral corticosteroid therapy in a subject having bullous pemphigoid are provided. In some embodiments, the method comprises:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject to be treated has (i) a baseline Bullous Pemphigoid Disease Area Index (BPDAI) activity score ≥24, and (ii) a baseline pruritus Numerical Rating Score (NRS) ≥4; and wherein the subject is on a background therapy comprising an oral corticosteroid.

In another aspect, methods for achieving sustained disease remission in the absence of oral corticosteroid therapy in a subject having bullous pemphigoid are provided. In some embodiments, the method comprises:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject to be treated has (i) a baseline Bullous Pemphigoid Disease Area Index (BPDAI) activity score ≥24, and (ii) a baseline pruritus Numerical Rating Score (NRS) 4.

In some embodiments, the subject is on a background therapy comprising an oral corticosteroid at the start of treatment with the IL-4R antagonist, and wherein the method further comprises discontinuing the oral corticosteroid therapy. In some embodiments, the subject to be treated has a baseline BPDAI activity score ≥24 and <60 and is on a background therapy of prednisone or prednisolone 0.5 mg/kg/day. In some embodiments, the subject to be treated has a baseline BPDAI activity score ≥60 and is on a background therapy of prednisone or prednisolone 0.75 mg/kg/day. In some embodiments, the subject was previously treated with an immunosuppressant.

In some embodiments, the subject is an adult.

In some embodiments, at least 8 doses of the IL-4R antagonist are administered to the subject.

In some embodiments, wherein treatment with the IL-4R antagonist results in:
achievement of sustained remission by week 36 from the start of treatment;
reduction in total cumulative dose of OCS administered from baseline to week 36 from the start of treatment;
reduction in weekly average of daily peak pruritus numerical rating score (NRS) from baseline to week 36 from the start of treatment;
reduction in BPDAI score from baseline to week 36 from the start of treatment; increase in time to first use of rescue medication from baseline to week 36 from the start of treatment;
reduction in BP180 autoantibody titer and/or BP230 autoantibody titer from baseline to week 36 from the start of treatment;
improvement in autoimmune bullous disease quality of life (ABQOL) from baseline to week 36 from the start of treatment; and/or
reduction in percent body surface area (BSA) of BP involvement from baseline to week 36 from the start of treatment.

In some embodiments, treatment with the IL-4R antagonist results in a reduction in BPDAI activity score of at least 50%, at least 75%, or at least 90% from baseline to week 36. In some embodiments, treatment with the IL-4R antagonist results in a reduction in BPDAI activity score of at least 50%, at least 75%, or at least 90% from baseline to week 52.

In some embodiments, treatment with the IL-4R antagonist results in achievement of sustained remission by week 36 from the start of treatment. In some embodiments, treatment with the IL-4R antagonist results in achievement of sustained remission by week 52 from the start of treatment.

In some embodiments, treatment with the IL-4R antagonist results in improvement (reduction) in Peak Pruritus NRS from baseline to week 36 from the start of treatment. In some embodiments, treatment with the IL-4R antagonist results in an improvement (reduction) of 4 points in Peak Pruritus NRS (e.g., weekly average of daily Peak Pruritus NRS) from baseline to week 36. In some embodiments, treatment with the IL-4R antagonist results in an improvement (reduction) of 4 points in Peak Pruritus NRS (e.g., weekly average of daily Peak Pruritus NRS) from baseline to week 52.

In some embodiments, treatment with the IL-4R antagonist results in a reduction in total cumulative dose of OCS administered from baseline to week 36 from the start of treatment.

In some embodiments, treatment with the IL-4R antagonist results in a reduction in total cumulative dose of OCS administered from baseline to week 52 from the start of treatment.

In some embodiments, the subject has a baseline Peak Pruritus NRS 7.

In some embodiments, the subject has had prior systemic corticosteroid use for BP.

In some embodiments, the IL-4R antagonist is administered to the subject as an initial dose followed by one or more secondary doses, wherein the initial dose is about 600 mg and each secondary dose is about 300 mg. In some embodiments, the secondary dose is administered two weeks after the immediately preceding dose (Q2W).

In some embodiments, the IL-4R antagonist is administered subcutaneously.

In some embodiments, the anti-IL-4R antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector. In some embodiments, the IL-4R antagonist is contained in a pen delivery device.

In another aspect, pharmaceutical compositions comprising an IL-4R antagonist for achieving sustained disease remission in the absence of oral corticosteroid therapy in a subject having bullous pemphigoid are provided.

In another aspect, pharmaceutical compositions comprising an IL-4R antagonist for reducing or eliminating the need for oral corticosteroid therapy in a subject having bullous pemphigoid are provided.

In another aspect, pharmaceutical compositions comprising an IL-4R antagonist for achieving sustained disease remission in the absence of oral corticosteroid therapy in a subject having bullous pemphigoid are provided.

In another aspect, provided herein are IL-4R antagonists for use in the preparation of a medicament for achieving sustained disease remission in the absence of oral corticosteroid therapy in a subject having bullous pemphigoid.

In another aspect, provided herein are IL-4R antagonists for use in the preparation of a medicament for reducing or eliminating the need for oral corticosteroid therapy in a subject having bullous pemphigoid.

In another aspect, provided herein are IL-4R antagonists for use in the preparation of a medicament for achieving sustained disease remission in the absence of oral corticosteroid therapy in a subject having bullous pemphigoid.

In another aspect, provided herein are methods for the treatment of an adult patient with bullous pemphigoid (BP). In some embodiments, the method comprises subcutaneously administering dupilumab to the patient as an initial dose of 600 mg (e.g., two 300 mg injections), followed by 300 mg given every other week (Q2W). In some embodiments, the patient has a baseline Bullous Pemphigoid Disease Area Index (BPDAI) activity score ≥24 on a scale of 0-360 and a weekly average Peak Pruritus NRS score 4 on a scale of 0-10. In some embodiments, the method further comprises administering a tapering course of oral corticosteroids.

In another aspect, provided herein is a pharmaceutical composition comprising dupilumab for use in the treatment of an adult patient with BP. In some embodiments, the pharmaceutical composition comprising dupilumab is for subcutaneous administration to the patient as an initial dose of 600 mg (e.g., two 300 mg injections), followed by subcutaneous administration of 300 mg Q2W. In some embodiments, the patient has a baseline Bullous Pemphigoid Disease Area Index (BPDAI) activity score ≥24 on a scale of 0-360 and a weekly average Peak Pruritus NRS score ≥4 on a scale of 0-10. In some embodiments, the pharmaceutical composition comprising dupilumab is for use in combination with a tapering course of oral corticosteroids.

In another aspect, provided herein is dupilumab for use in the preparation of a medicament for the treatment of an adult patient with BP. In some embodiments, the dupilumab is for subcutaneous administration to the patient as an initial dose of 600 mg (e.g., two 300 mg injections), followed by subcutaneous administration of 300 mg Q2W. In some embodiments, the patient has a baseline Bullous Pemphigoid Disease Area Index (BPDAI) activity score ≥24 on a scale of 0-360 and a weekly average Peak Pruritus NRS score ≥4 on a scale of 0-10. In some embodiments, the dupilumab is for use in combination with a tapering course of oral corticosteroids.

In another aspect, provided herein are methods for the treatment of an adult patient with bullous pemphigoid (BP) who is uncontrolled on systemic corticosteroid(s). In some embodiments, the method comprises subcutaneously administering dupilumab to the patient as an initial dose of 600 mg (e.g., two 300 mg injections), followed by 300 mg given every other week (Q2W). In some embodiments, the patient has a baseline Bullous Pemphigoid Disease Area Index (BPDAI) activity score ≥24 on a scale of 0-360 and a weekly average Peak Pruritus NRS score 4 on a scale of 0-10.

In another aspect, provided herein is a pharmaceutical composition comprising dupilumab for use in the treatment of an adult patient with BP who is uncontrolled on systemic corticosteroid(s). In some embodiments, the pharmaceutical composition comprising dupilumab is for subcutaneous administration to the patient as an initial dose of 600 mg (e.g., two 300 mg injections), followed by subcutaneous administration of 300 mg Q2W. In some embodiments, the patient has a baseline Bullous Pemphigoid Disease Area Index (BPDAI) activity score ≥24 on a scale of 0-360 and a weekly average Peak Pruritus NRS score ≥4 on a scale of 0-10.

In another aspect, provided herein is dupilumab for use in the preparation of a medicament for the treatment of an adult patient with BP who is uncontrolled on systemic corticosteroid(s). In some embodiments, the dupilumab is for subcutaneous administration to the patient as an initial dose of 600 mg (e.g., two 300 mg injections), followed by subcutaneous administration of 300 mg Q2W. In some embodiments, the patient has a baseline Bullous Pemphigoid Disease Area Index (BPDAI) activity score ≥24 on a scale of 0-360 and a weekly average Peak Pruritus NRS score ≥4 on a scale of 0-10.

Other embodiments will be apparent from a review of the ensuing detailed description.

### BRIEF DESCRIPTION OF THE FIGURES

FIG. 1. Overview of clinical trial study design disclosed in Example 1. *Loading 600 mg dose of dupilumab (or equivalent placebo dose) given at day 1. Note: Tapering of prednisone (or prednisolone) should begin no later than W6. ↑ Moderate BP patients may be escalated to 0.75 mg/kg/day if patient is not showing signs of control of disease activity by week 2. Abbreviations: BL, Baseline; EP, endpoint; q2w, every 2 weeks; SC, subcutaneous.

FIG. 2. A significantly higher proportion of dupilumab-treated patients achieved sustained BP disease remission at Week 36. N=number of patients with observed data for the endpoint; n=number of patients with observed responder status for the endpoint. Multiple imputation was used for patients without observed data. ^{o}Components of sustained disease remission were prespecified for analysis in the statistical analysis plan. They were not prespecified for inclusion in the statistical hierarchy and are therefore nominal. ^{b}The disease relapse component was only evaluated among patients who completed the OCS taper by Week 16.

FIG. 3. Treatment with dupilumab significantly improved itch, as measured by percent change from baseline in Peak Pruritus Numerical Rating Scale (PP-NRS) score. *Week 17 is nominally significant at p<0.05. Weeks 18-35 (inclusive) are nominally significant at p<0.01. Week 36 is significant at p<0.01. n=number of patients with observed responder status for the endpoint. Multiple imputation was used for patients with observed data. LS, least squares; OCS, oral corticosteroids; SE, standard error.

FIG. 4. Treatment with dupilumab significantly improved BP disease activity, as measured by percent change from baseline in Bullous Pemphigoid Disease Area Index (BPDAI) activity score. *Weeks 16, 24, and 32 are nominally significant at p<0.01. Week 36 is significant at p<0.01. n=number of patients with observed responder status for the endpoint. Multiple imputation was used for patients with observed data. LS, least squares; OCS, oral corticosteroids; SE, standard error.

FIG. 5. Subjects treated with dupilumab were more likely to discontinue OCS and remain off OCS. CI, confidence interval; n, number of patients at risk; OCS, oral corticosteroids.

FIG. 6. Improvements in ABQOL favored dupilumab over placebo at Week 36. At baseline, ABQOL mean (SD) was 21.7 (9.1) for placebo and 22.9 (9.8) for dupilumab. At Week 36, LS mean difference vs placebo (95% CI) was −3.70 (−7.346, −0.061). n=number of patients with observed or imputed data.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

As used herein, “bullous pemphigoid” or “BP” refers to an autoimmune subepidermal blistering disease characterized by large, tense, serous, or hemorrhagic bullae arising on erythematous, urticarial, or eczematous skin.

As used herein, the term “subject in need thereof” refers to a human or a non-human animal having bullous pemphigoid (BP). In some embodiments, the subject has a confirmed diagnosis of BP based on histopathology, immunopathology, and/or serology. In some embodiments, the term “a subject in need thereof” refers to an adult patient who is 50 years of age, e.g., ≥60 years of age, ≥65 years of age, ≥70 years of age, ≥75 years of age, or ≥80 years of age. The terms “subject” and “patient” are used interchangeably herein.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, provided herein are pharmaceutical compositions and methods for treating bullous pemphigoid (BP) or reducing or ameliorating one or more symptoms of BP in a subject in need thereof, comprising administering to the subject an interleukin-4 receptor (IL-4R) antagonist, such as an anti-IL-4Rα antibody or antigen-binding fragment thereof.

In another aspect, provided herein are pharmaceutical compositions and methods for improving one or more BP-associated parameters or patient-recorded outcome (PRO) measures in a subject in need thereof, comprising administering to the subject an IL-4R antagonist, such as an anti-IL-4Rα antibody or antigen-binding fragment thereof.

In another aspect, provided herein are pharmaceutical compositions and methods for reducing or eliminating the need for background therapy, such as oral corticosteroid therapy, in a subject having bullous pemphigoid, comprising administering to the subject an IL-4R antagonist, such as an anti-IL-4Rα antibody or antigen-binding fragment thereof. In some embodiments, the subject is on a background therapy comprising an oral corticosteroid.

In yet another aspect, provided herein are pharmaceutical compositions and methods for achieving sustained disease remission in the absence of oral corticosteroid therapy in a subject having bullous pemphigoid, comprising administering to the subject an IL-4R antagonist, such as an anti-IL-4Rα antibody or antigen-binding fragment thereof. In some embodiments, the subject is on a background therapy comprising an oral corticosteroid at the start of treatment with the IL-4R antagonist, and the method comprises discontinuing the oral corticosteroid therapy.

In some embodiments, a subject to be treated is an adult. In some embodiments, the subject is ≥20 years of age, ≥30 years of age, ≥40 years of age, ≥50 years of age, ≥60 years of age, or ≥70 years of age. In some embodiments, the subject is 12 years of age. In some embodiments, the subject is an adolescent. In some embodiments, the subject is a pediatric subject <18 years of age, e.g., <12 years of age.

In some embodiments, a subject to be treated according to the methods disclosed herein has a history of documented diagnosis of BP based on histopathology, immunopathology, and/or serology. In some embodiments, the subject has a history of direct immunofluorescence on a skin biopsy specimen demonstrating linear IgG and/or C3 at the basement membrane zone. In some embodiments, the subject has a history of indirect immunofluorescence demonstrating IgG antibodies localizing to the roof (epidermal side) of split-skin substrate.

In some embodiments, the subject has an elevated IgG serum antibody level for BPAG1 (BP230) and/or BPAG2 (BP180). In some embodiments, the subject has a baseline IgG serum antibody level for BP180 and/or BP230 of at least 20 U/mL, 30 U/mL, 40 U/mL, 50 U/mL, 60 U/mL, 70 U/mL 80 U/mL, 90 U/mL, 100 U/mL, 125 U/mL, 150 U/mL, 175 U/mL, 200 U/mL, 225 U/mL, or 250 U/mL, as measured by an immunoassay (e.g., ELISA).

In some embodiments, a subject to be treated does not have Brunsting-Perry cicatricial pemphigoid, anti-p200 pemphigoid, epidermolysis bullosa acquisita, or BP with concomitant pemphigus vulgaris.

In some embodiments, a subject to be treated has a baseline Bullous Pemphigoid Disease Area Index (BPDAI) activity score ≥24. In some embodiments, the subject has a baseline BPDAI activity score ≥30, ≥35, ≥40, ≥45, ≥50, ≥55, ≥60, ≥65, ≥70, ≥75, ≥80, ≥85, ≥90, ≥95, or ≥100. In some embodiments, the subject has a baseline BPDAI activity score ≥24 and <60, or ≥30 and <60, or ≥36 and <60.

In some embodiments, a subject to be treated has a baseline Bullous Pemphigoid Disease Area Index (BPDAI) pruritus score ≥10. In some embodiments, the subject has a baseline BPDAI pruritus score ≥12, ≥15, ≥18, or ≥20.

In some embodiments, a subject to be treated has a baseline pruritus Numerical Rating Score (NRS) ≥4. In some embodiments, the subject has a baseline pruritus NRS ≥5, ≥6, or ≥7.

In some embodiments, a subject to be treated has an elevated baseline level of one or more markers of Type 2 inflammation, such as but not limited to total IgE, TARC, PARC, and blood eosinophils. In some embodiments, the subject has an elevated baseline serum total IgE.

In some embodiments, the subject has an elevated baseline serum TARC. In some embodiments, the subject has an elevated baseline serum PARC. In some embodiments, the subject has an elevated baseline blood eosinophil level.

In some embodiments, the subject has a baseline total IgE level of at least 150 U/mL, at least 200 U/mL, at least 300 U/mL, at least 400 U/mL, at least 500 U/mL, at least 600 U/mL, at least 700 U/mL, at least 800 U/mL, at least 900 U/mL, at least 1000 U/mL, at least 1250 U/mL, at least 1500 U/mL, or at least 1750 U/mL, e.g., as measured using the IMMUNOCAP® assay [Phadia, Inc. Portage, Ml]).

In some embodiments, a subject to be treated has a history of prior treatment with corticosteroids (e.g., oral corticosteroids such as but not limited to prednisone or prednisolone, or topical corticosteroids such as clobetasol propionate). In some embodiments, a subject to be treated has a history of prior treatment with a systemic immunosuppressant (such as but not limited to azathioprine, cyclophosphamide, cyclosporine, mycophenolate mofetil, methotrexate, or rituximab). In some embodiments, a subject to be treated has a history of prior treatment with intravenous immunoglobulin (IVIG). In some embodiments, a subject to be treated is a subject who is non-responsive, inadequately responsive, intolerant, or resistant to treatment with an oral corticosteroid, a topical corticosteroid, a systemic immunosuppressant, and/or IVIG. In some embodiments, a subject to be treated has a contraindication for an oral corticosteroid, a topical corticosteroid, a systemic immunosuppressant, and/or IVIG.

In some embodiments, a subject to be treated is on a background therapy. In some embodiments, the subject is on a background therapy comprising a corticosteroid, e.g., an oral corticosteroid such as prednisone or prednisolone. In some embodiments, the subject is on a background therapy comprising a topical treatment (e.g., a topical corticosteroid, a topical calcineurin inhibitor, or topical crisaborole).

In some embodiments wherein the subject is on a background therapy (e.g., a background therapy comprising an oral corticosteroid) at the start of treatment with the IL-4R antagonist, the method further comprises discontinuing or tapering the background therapy (e.g., oral corticosteroid therapy). In some embodiments, the subject is on a tapering course of oral corticosteroids. In some embodiments, the background therapy (e.g., oral corticosteroid therapy) is discontinued or tapered over the course of 2 weeks, 4 weeks, 6 weeks, 8 weeks, 10 weeks, 12 weeks, or longer. In some embodiments, the background therapy (e.g., oral corticosteroid therapy) is discontinued or tapered within 16 weeks from the start of treatment with the IL-4R antagonist. In some embodiments, the discontinuing or tapering of background therapy is initiated once the subject exhibits control of disease activity, e.g., sustained control of disease activity for at least 2 weeks. In some embodiments, “control of disease activity” occurs when new lesions (e.g., blisters, urticarial plaques) cease to form and existing lesions begin to heal (e.g., show signs of epithelialization) (Murrell, et al., J Am Acad Dermatol 2012, 66:479-485). In some embodiments, the background therapy (e.g., oral corticosteroid therapy) is tapered over regular intervals (e.g., every week, every two weeks, every three weeks, every four weeks, or every month) by reducing the dose of the background therapy by about one-half, one-third, or one-quarter compared to the previous dose. See, e.g., Venning, et al., Br J Dermatol 2012, 167:1200-1214. In some embodiments, the background therapy is tapered as disclosed in the Examples section below. For example, in some embodiments, the subject is on a background therapy comprising prednisone or prednisolone at a dose of 0.5 mg/kg/day, and the discontinuing or tapering comprises administering to the subject prednisone or prednisolone at a dose of 0.3 mg/kg/day for 2 weeks, followed by prednisone or prednisolone at a dose of 0.2 mg/kg/day for 2 weeks, followed by prednisone or prednisolone at a dose of 0.1 mg/kg/day for 2 weeks, followed by prednisone or prednisolone at a dose of 0.05 mg/kg/day for 2 weeks, followed by discontinuation of the prednisone or prednisolone. In some embodiments, the subject is on a background therapy comprising prednisone or prednisolone at a dose of 0.75 mg/kg/day, and the discontinuing or tapering comprises administering to the subject prednisone or prednisolone at a dose of 0.5 mg/kg/day for 2 weeks, followed by prednisone or prednisolone at a dose of 0.3 mg/kg/day for 2 weeks, followed by prednisone or prednisolone at a dose of 0.2 mg/kg/day for 2 weeks, followed by prednisone or prednisolone at a dose of 0.1 mg/kg/day for 2 weeks, followed by prednisone or prednisolone at a dose of 0.05 mg/kg/day for 2 weeks, followed by discontinuation of the prednisone or prednisolone.

In some embodiments, a subject to be treated has, or has had, at least one comorbidity. In some embodiments, a subject to be treated has, or has had, a concomitant type 2 inflammatory condition. As used herein, a “type 2 inflammation condition” is a disease, disorder, or condition associated with a T helper 2 (TH2)-mediated immune response (Gandhi, et al., Nat Rev Drug Discov., 2016, 15(1):35-50). Non-limiting examples of type 2 inflammatory conditions include asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), atopic conjunctivitis, atopic dermatitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), chronic eosinophilic pneumonia (CEP) and exercise induced bronchospasm. In some embodiments, the subject has a concomitant atopic disease or condition selected from the group consisting of food allergy, atopic dermatitis, asthma, chronic rhinosinusitis, allergic rhinitis, or allergic conjunctivitis. In some embodiments, the subject does not have a concomitant atopic disease or condition.

In some embodiments, a subject to be treated is selected on the basis of exhibiting one or more inclusion criteria disclosed in Example 1. In some embodiments, a subject to be treated is further selected on the basis of not exhibiting one or more exclusion criteria disclosed in Example 1.

### BP-Related Parameters

In some embodiments, the therapeutic methods disclosed herein result in an improvement in one or more endpoints or BP-related parameters that are used to assess the presence or severity of BP in a subject. Examples of BP-related parameters include, but are not limited to: (a) improvement in BP clinical symptoms, such as healing of existing lesions (e.g., showing signs of epithelialization) or decrease in number of blisters and lesions; (b) achievement of complete remission (defined herein as absence of new lesions and epithelialization of old lesions; (c) absence of disease relapse; (d) absence of need for rescue therapy or increase in time to first use of rescue medication; (d) reduction or elimination of background therapy (e.g., oral corticosteroid therapy), e.g., as measured by total cumulative dose of background therapy during a defined period; (e) duration of complete remission while not requiring background therapy (e.g., oral corticosteroid therapy); (f) change (e.g., reduction) in frequency and/or intensity of symptoms, e.g., as measured using the Bullous Pemphigoid Disease Area Index (BPDAI) activity score, the BPDAI pruritus score, the BSA assessment tool, the Pruritus Numerical Rating Scale (NRS), the Skin Pain NRS, the Sleep NRS, the Patient Global Assessment of Disease Severity (PGADS), the Patient Global Assessment of Treatment (PGAT), the European Quality of Life 5-Dimension 5-Level Scale (EQ-5D-5L), or the Autoimmune Bullous Disease Quality of Life (ABQOL); or (g) improvement in one or more biomarkers associated with BP, such as but not limited to total IgE, TARC, PARC, autoantibody IgG against BP180, BP230, IgE titers against BP180 and BP230, and IgG4 titers against BP180 and BP230. Methods for assessing these and other BP-related parameters are described in the Examples section below.

To determine whether a BP-related parameter has “improved,” the parameter is quantified at baseline (e.g., prior to the start of treatment with the IL-4R antagonist) and at one or more timepoints after administration of the IL-4R antagonist. For example, a BP-related parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 30,, week 32, week 34, week 36, week 38, week 40, week 42, week 44, week 46, week 48, week 50, week 52, or longer, after the initial treatment with a pharmaceutical composition of the present disclosure. The difference between the value of the parameter at a particular timepoint following initiation of treatment and the value of the parameter at baseline is used to establish whether there has been an improvement in the BP-related parameter.

In some embodiments, treatment of a subject with an IL-4R antagonist (e.g., an anti-IL-4R antibody) results in an improvement in BP clinical symptoms, such as the epithelialization of existing lesions or a decrease in the quantity, size, and/or severity of blisters and lesions. In some embodiments, treatment with an IL-4R antagonist results in an increase in epithelialization of existing lesions of at least 10%, 15%, 20%, 25%, 30%, 35%, 40%, 45%, 50% or more relative to baseline. In some embodiments, treatment with an IL-4R antagonist results in a decrease of at least 10%, 15%, 20%, 25%, 30%, 35%, 40%, 45%, 50% or more in the quantity, size, and/or severity of blisters and lesions, relative to baseline. In some embodiments, the change in BP clinical symptoms is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in one or more BP-associated patient-recorded outcome (PRO) measures. In some embodiments, the BP-associated PRO is the BPDAI activity score, the BPDAI pruritus score, the BSA assessment tool, the Pruritus NRS, the Skin Pain NRS, the Sleep NRS, the PGADS, the PGAT, the EQ-5D-5L, or the ABQOL.

In some embodiments, treatment of a subject with an IL-4R antagonist (e.g., an anti-IL-4R antibody) results in an improvement in BPDAI activity score. The BPDAI activity score is the arithmetic sum of 3 subcomponents: cutaneous blisters/erosions, cutaneous urticaria/erythema, and mucosal blisters/erosions. For each of the cutaneous blisters/erosions and cutaneous urticaria/erythema components, a score from 0-10 (0=absent; 10=>3 lesions, and least one lesion >5 cm diameter or entire area) is assigned for each of the following regions: head, neck, chest, left arm, right arm, hands, abdomen, genitals, back/buttocks, left leg, right leg, and feet. For the mucosal blisters/erosions subcomponent, a score from 0-10 (0=absent; 10=entire area) is assigned for each of the following regions: eyes, nose, buccal mucosa, hard palate, soft palate, upper gingiva, lower gingiva, tongue, floor of mouth, labial mucosa, posterior pharynx, and anogenital. Scores can range from 0 to 360 for BPDAI total activity (maximum 240 for total cutaneous activity [maximum 120 for blisters/erosions and maximum 120 for urticaria/erythema] and 120 for mucosal activity), with higher scores indicating greater disease activity. In some embodiments, treatment with an IL-4R antagonist results in a decrease in BPDAI activity score of at least 10%, 15%, 20%, 25%, 30%, 35%, 40%, 45%, 50% or more relative to baseline (e.g., a subject's BPDAI activity score prior to the onset of treatment). In some embodiments, the change in BPDAI activity score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in BPDAI pruritus score. For the BPDAI pruritus score, the intensity of pruritus is subjectively graded by the subject using a visual analog scale from 0-10 (where 0 represents no itch and 10 represents maximal itching), to indicate severity of itching (i) in the past 24 hours, (ii) in the past week, and (iii) in the past month, producing a total score out of a maximum possible score of 30. In some embodiments, treatment with an IL-4R antagonist results in a decrease in BPDAI pruritus score of at least 10%, 15%, 20%, 25%, 30%, 35%, 40%, 45%, 50% or more relative to baseline (e.g., a subject's BPDAI pruritus score prior to the onset of treatment). In some embodiments, the change in BPDAI pruritus score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in a reduction in BPDAI activity score of at least 50%, at least 75%, or at least 90%, e.g., relative to baseline (e.g., a subject's BPDAI pruritus score prior to the onset of treatment). In some embodiments, the reduction in BPDAI pruritus score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in Pruritus NRS. For Pruritus NRS, the subject grades the intensity of their pruritus (itch) during a 24-hour recall period on a scale of 0 to 10, in which 0 indicates no itch while 10 indicates worst itch possible. In some embodiments, treatment with an IL-4R antagonist results in a decrease in Pruritus NRS score of at least 10%, 15%, 20%, 25%, 30%, 35%, 40%, 45%, 50% or more relative to baseline (e.g., a subject's Pruritus NRS score prior to the onset of treatment). In some embodiments, the change in Pruritus NRS score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In treatment of a subject with an IL-4R antagonist (e.g., an anti-IL-4R antibody) results in an improvement in skin pain NRS. For skin pain NRS, the subject grades the intensity of their skin pain on a scale of 0 to 10, in which 0 indicates no pain while 10 indicates worst pain possible. In some embodiments, treatment with an IL-4R antagonist results in a decrease in skin pain NRS score of at least 10%, 15%, 20%, 25%, 30%, 35%, 40%, 45%, 50% or more relative to baseline (e.g., a subject's skin pain NRS score prior to the onset of treatment). In some embodiments, the change in skin pain NRS score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in Sleep NRS. For sleep NRS, the subject grades the quality of their sleep on a scale of 0 to 10, in which 0 indicates worst possible sleep while 10 indicates best possible sleep. In some embodiments, treatment with an IL-4R antagonist results in an increase in sleep NRS score of at least 10%, 15%, 20%, 25%, 30%, 35%, 40%, 45%, 50% or more relative to baseline (e.g., a subject's sleep NRS score prior to the onset of treatment). In some embodiments, the change in sleep NRS score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in PGADS. For the PGADS score, the subject rates their overall bullous pemphigoid symptoms based on a 5-point scale (from “no symptoms” to “very severe symptoms”). In some embodiments, treatment with an IL-4R antagonist results in a decrease in PGADS score of at least 10%, 15%, 20%, 25%, 30%, 35%, 40%, 45%, 50% or more relative to baseline (e.g., a subject's PGADS score prior to the onset of treatment). In some embodiments, the change in PGADS score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in PGAT. For the PGAT score, the subject rates their satisfaction with the study treatment based on a 5-point scale (from “much better” to “much worse”). In some embodiments, treatment with an IL-4R antagonist results in a decrease in PGAT score of at least 10%, 15%, 20%, 25%, 30%, 35%, 40%, 45%, 50% or more relative to baseline (e.g., a subject's PGAT score prior to the onset of treatment). In some embodiments, the change in PGAT score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in EQ-5D-5L. The EQ-5D-5L is a standardized questionnaire used to assess health status (Brooks, 1996) (Rabin, et al., Value Health, 2014, 17:70-76). It consists of a descriptive system and the EQ visual analogue scale (EQ VAS). The descriptive system comprises the following 5 dimensions: mobility, self-care, usual activities, pain/discomfort, and anxiety/depression. For each dimension, participants select one of 5 levels: no problems, slight problems, moderate problems, severe problems, and extreme problems. The EQ VAS records the participant's self-rated health on a vertical visual analogue scale where the endpoints are labelled ‘Best imaginable health state’ and ‘Worst imaginable health state’. In some embodiments, treatment with an IL-4R antagonist results in an improvement in EQ-5D-5L score of at least 10%, 15%, 20%, 25%, 30%, 35%, 40%, 45%, 50% or more relative to baseline. In some embodiments, the change in EQ-5D-5L score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in ABQOL. The ABQOL questionnaire consists of 17 items, which encompass physical burden of the disease, psychiatric effects, and effects on daily life functioning. Each question ranges from 0 to 3 points, with higher scores indicating poorer quality of life, and a maximum possible score of 51. In some embodiments, treatment with an IL-4R antagonist results in an improvement in ABQOL score of at least 10%, 15%, 20%, 25%, 30%, 35%, 40%, 45%, 50% or more relative to baseline. In some embodiments, the change in ABQOL score is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in the percentage of body surface area (BSA) affected by BP. BSA is assessed for each major section of the body (head and neck, anterior trunk, back, upper limbs, lower limbs, and genitals) and reported as a percentage of all major body sections combined. In some embodiments, treatment with an IL-4R antagonist results in a decrease in the percentage of overall BSA affected by BP of at least 10%, 15%, 20%, 25%, 30%, 35%, 40%, 45%, 50% or more relative to baseline. In some embodiments, treatment with an IL-4R antagonist results in a decrease in the percentage of BSA affected by BP of at least 10%, 15%, 20%, 25%, 30%, 35%, 40%, 45%, 50% or more relative to baseline for one or more body sections (e.g., one or more of head and neck, anterior trunk, back, upper limbs, lower limbs, and genitals). In some embodiments, the change in BSA is measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in an improvement in or normalization of one or more BP-associated biomarkers. Non-limiting examples of BP-associated biomarkers include anti-BP180 autoantibodies, anti-BP230 autoantibodies, total IgE, TARC/CCL17, and PARC/CCL18. In some embodiments, the BP-associated biomarker is determined using a tissue sample from the subject (e.g., skin biopsy samples). In some embodiments, treatment of a subject with an IL-4R antagonist results in a normalization of one or more BP-associated biomarkers relative to baseline (e.g., a subject's level of expression of the BP-associated biomarker), e.g., as measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist.

In some embodiments, treatment of a subject with an IL-4R antagonist (e.g., an anti-IL-4R antibody) results in complete remission, e.g., as measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist. In some embodiments, “complete remission” is defined as the absence of new lesions and the epithelialization of old lesions. In some embodiments, treatment of a subject with an IL-4R antagonist (e.g., an anti-IL-4R antibody) results in sustained disease remission, e.g., as measured at day 8, 15, 22, 25, 29, 36, 43, 50, 57, 64, 71, 85, 113 or later following the initial administration of the IL-4R antagonist, or after 2, 4, 6, 8, 10, 12, 14, 16, 18, 20, 22, 24, 26, 28, 30, 32, 34, 36, 38, 40, 42, 44, 46, 48, 50, or 52 weeks of treatment with the IL-4R antagonist. In some embodiments, “sustained remission” is defined as the achievement of complete remission, plus the elimination of any concomitant background therapy (e.g., OCS, e.g., by no later than week 16 from the start of treatment), plus the absence of disease relapse and/or absence of need for rescue therapy following the elimination of the background therapy (e.g., OCS).

In some embodiments, the IL-4R antagonist (e.g., an anti-IL-4R antibody such as dupilumab) is administered to the subject in combination with a tapering course of oral corticosteroids until disease control has occurred in the subject. In some embodiments, the IL-4R antagonist is subsequently administered as a monotherapy. In some embodiments, the IL-4R antagonist is subsequently administered as a monotherapy until a relapse (i.e., loss of control of disease activity) occurs, in which case corticosteroids may be added if medically advisable. In some embodiments, “disease control” is measured by whether new lesions cease to form and existing lesions begin to heal.

### Interleukin-4 Receptor Antagonists

In some embodiments, the methods of the present disclosure comprise administering to a subject in need thereof (e.g., a subject having BP) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4-and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified. In some embodiments, the antibody is a human IgG antibody. In some embodiments, the antibody is a human IgG4 antibody.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L}domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}—C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homodimer or heterodimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein, et al., 2012, mAbs, 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor, et al., (1992) Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, NJ). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

The antibodies used in the methods of the present disclosure may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc.

In some embodiments, an antibody (such as an anti-IL-4R antibody as disclosed herein) does not have a C-terminal lysine in the heavy chain. For example, a C-terminal lysine that is present at the end of a heavy chain sequence may be removed as a post-translational modification during manufacture, e.g., during protein expression. Alternatively, a C-terminal lysine may be removed by recombinant technology (e.g., the coding sequence of the heavy chain does not include a codon for a C-terminal lysine). Thus, contemplated within the present disclosure are antibodies comprising a heavy chain in which a C-terminal lysine, if included in the amino acid sequence (e.g., as in SEQ ID NO:366), is absent (e.g., as in SEQ ID NO:9).

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence GFTFRDYA (SEQ ID NO:3), the HCDR2 comprises the amino acid sequence ISGSGGNT (SEQ ID NO:4), the HCDR3 comprises the amino acid sequence AKDRLSITIRPRYYGLDV (SEQ ID NO:5), the LCDR1 comprises the amino acid sequence QSLLYSIGYNY (SEQ ID NO:6), the LCDR2 comprises the amino acid sequence LGS, and the LCDR3 comprises the amino acid sequence MQALQTPYT (SEQ ID NO:8).

In some embodiments, the anti-IL-4R antibody or antigen-binding fragment thereof comprises an HCDR1 comprising the amino acid sequence GFTFRDYA (SEQ ID NO:3), an HCDR2 comprising the amino acid sequence ISGSGGNT (SEQ ID NO:4), an HCDR3 comprising the amino acid sequence AKDRLSITIRPRYYGLDV (SEQ ID NO:5), an LCDR1 comprising the amino acid sequence QSLLYSIGYNY (SEQ ID NO:6), an LCDR2 comprising the amino acid sequence LGS, and an LCDR3 comprising the amino acid sequence MQALQTPYT (SEQ ID NO:8), and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10. In some embodiments, the anti-IL-4R antibody comprises a heavy chain comprising the amino acid sequence of SEQ ID NO:9 and a light chain comprising the amino acid sequence of SEQ ID NO:10.

An exemplary anti-IL-4R antibody is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), BA2101, comekibart, manfidokimab, LQ036, MEDI 9314, NS-402, QX-005N, rademikibart, stapokibart, TQH-2722, or any of the anti-IL-4Rα antibodies as set forth in U.S. Pat. Nos. 7,186,809, 7,605,237, 7,638,606, 8,092,804, 8,679,487, 8,877,189, 11,667,717, 11,725,057, 10,774,141, 11,897,960, 11,939,387, International Patent Publication Nos. WO2020/096381, WO 2020/135471, WO 2020/135710, WO 2020/182197, WO2020/239134, WO 2021/213329, WO2022/052974, WO2022/136669, or WO2022/136675, or US Patent Publication Nos. 2021/0238294, 2022/0073631, 2022/0162328, 2022/0411519, 2023/0105029, or 2023/0295312, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 1 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55). In some embodiments, the anti-IL-4Rα antibody comprises an LCVR/HCVR sequence pair selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL). In some embodiments, the anti-IL-4Rα antibody comprises the HCVR/LCVR sequence pair of AJOU-83-VH/AJOU-90-VL.

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171). In some embodiments, the anti-IL-4Rα antibody comprises an HCVR/LCVR sequence pair selected from the group consisting of REGN-VH-3/REGN-VL-11; REGN-VH-19/REGN-VL-27; REGN-VH-35/REGN-VL-43; REGN-VH-51/REGN-VL-59; REGN-VH-67/REGN-VL-75; REGN-VH-83/REGN-VL-91; REGN-VH-99/REGN-VL-107; REGN-VH-115/REGN-VL-123; REGN-VH-147/REGN-VL-155; and REGN-VH-163/REGN-VL-171.

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-VH), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-C27-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-VL), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-C27-56-56-VL), SEQ ID NO:233 (STSA-C27-78-78-VL), SEQ ID NO:234 (STSA-C27-82-58-VL), SEQ ID NO:235 (STSA-C27-54-54-VL), SEQ ID NO:236 (STSA-C27-36-36-VL), SEQ ID NO:237 (STSA-C27-53-53-VL), SEQ ID NO:238 (STSA-C27-67-67-VL), SEQ ID NO:239 (STSA-C27-55-55-VL), SEQ ID NO:240 (STSA-C27-59-59-VL), SEQ ID NO:241 (STSA-C27-58-58-VL), SEQ ID NO:242 (STSA-C27-52-52-VL), or SEQ ID NO:243 (STSA-C27-Y2-Y2-VL). In some embodiments, the anti-IL-4Rα antibody comprises an HCVR/LCVR sequence pair selected from the group consisting of SEQ ID NOs:208/226 (STSA-C27), SEQ ID NOs:209/227 (STSA-C27-6-33), SEQ ID NOs:210/228 (STSA-C27-7-33), SEQ ID NOs:211/229 (STSA-C27-24-56), SEQ ID NOs:212/230 (STSA-C27-47-56), SEQ ID NOs:213/231 (STSA-C27-33-33), SEQ ID NOs:214/232 (STSA-C27-56-56), SEQ ID NOs:215/233 (STSA-C27-78-78), SEQ ID NOs:216/234 (STSA-C27-82-58), SEQ ID NOs:217/235 (STSA-C27-54-54), SEQ ID NOs:218/236 (STSA-C27-36-36), SEQ ID NOs:219/237 (STSA-C27-53-53), SEQ ID NOs:220/238 (STSA-C27-67-67), SEQ ID NOs:221/239 (STSA-C27-55-55), SEQ ID NOs:222/240 (STSA-C27-59-59), SEQ ID NOs:223/241 (STSA-C27-58-58), SEQ ID NOs:224/242 (STSA-C27-52-52), or SEQ ID NOs:225/243 (STSA-C27-Y2-Y2).

In some embodiments, an anti-IL-4Rα antibody comprises (i) an HCVR comprising the amino acid sequence of SEQ ID NO:244 (Y0188-1 VH), SEQ ID NO:245 (Y0188-2 VH), SEQ ID NO:246 (Y0188-3 VH), SEQ ID NO:247 (Y0188-4 VH), SEQ ID NO:248 (Y0188-6 VH), SEQ ID NO:249 (Y0188-8 VH), SEQ ID NO:250 (Y0188-9 VH), SEQ ID NO:251 (Y0188-10 VH), SEQ ID NO:252 (Y0188-14 VH), SEQ ID NO:253 (HV3-15-14 VH), SEQ ID NO:254 (HV3-48-14 VH), SEQ ID NO:255 (HV3-73*2-14 VH), SEQ ID NO:256 (HV3-72-14 VH), SEQ ID NO:257 (Y01-14 VH), SEQ ID NO:258 (162-14 VH), or SEQ ID NO:259 (VH73-14 VH); and (ii) an LCVR comprising the amino acid sequence of SEQ ID NO:260 (Y0188-1 VL), SEQ ID NO:261 (Y0188-2 VL), SEQ ID NO:262 (Y0188-3 VL), SEQ ID NO:263 (Y0188-4 VL), SEQ ID NO:264 (Y0188-6 VL), SEQ ID NO:265 (Y0188-8 VL), SEQ ID NO:266 (Y0188-9 VL), SEQ ID NO:267 (Y0188-10 VL), SEQ ID NO:268 (Y0188-14 VL), SEQ ID NO:269 (Y01-14 VL), SEQ ID NO:270 (164-14 VL), SEQ ID NO:271 (KV4-14 VL), SEQ ID NO:272 (KV1-27-14 VL), SEQ ID NO:273 (KV1-9-14 VL), SEQ ID NO:274 (KV1-NL1-14 VL), or SEQ ID NO:275 (KV1 D-43-14 VL). In some embodiments, the anti-IL-4Rα antibody comprises an HCVR/LCVR sequence pair selected from the group consisting of SEQ ID NOs:244/260 (Y0188-1), SEQ ID NOs:245/261 (Y0188-2), SEQ ID NOs:246/262 (Y0188-3), SEQ ID NOs:247/263 (Y0188-4), SEQ ID NOs:248/264 (Y0188-6), SEQ ID NOs:249/265 (Y0188-8), SEQ ID NOs:250/266 (Y0188-9), SEQ ID NOs:251/267 (Y0188-10), SEQ ID NOs:252/268 (Y0188-14), SEQ ID NOs:257/269 (Y01-14), and SEQ ID NOs:258/270 (162-14).

In some embodiments, an anti-IL-4Rα antibody comprises (i) an HCVR comprising the amino acid sequence of SEQ ID NO:276 (1A6 VH), SEQ ID NO:278 (1D8 VH), SEQ ID NO: 280 (1H9 VH), SEQ ID NO:282 (2H1 VH), SEQ ID NO:284 (2F8 VH), SEQ ID NO:286 (9B4 VH), SEQ ID NO:288 (9E7 VH), SEQ ID NO:290 (24G10 VH), SEQ ID NO:292 (25D6 VH), SEQ ID NO:294 (25G9 VH), SEQ ID NO:296 (35A7-1 VH), SEQ ID NO:298 (31B9 VH), SEQ ID NO:300 (34A2 VH), SEQ ID NO:302 (34H11 VH), SEQ ID NO:304 (35D5 VH), SEQ ID NO:306 (35A7-2 VH), SEQ ID NO:308 (36F4 VH), SEQ ID NO:310 (VH1021), SEQ ID NO:311 (VH1022), SEQ ID NO:312 (VH1023), SEQ ID NO:313 (VH1024), SEQ ID NO:314 (VH1025), SEQ ID NO:315 (VH1026), SEQ ID NO:316 (VH1027), or SEQ ID NO:317 (VH1028); and (ii) an LCVR comprising the amino acid sequence of SEQ ID NO:277 (1A6 VL), SEQ ID NO:279 (1 D8 VL), SEQ ID NO:281 (1H9 VL), SEQ ID NO:283 (2H1 VL), SEQ ID NO:285 (2F8 VL), SEQ ID NO:287 (9B4 VL), SEQ ID NO:289 (9E7 VL), SEQ ID NO:291 (24G10 VL), SEQ ID NO:293 (25D6 VL), SEQ ID NO:295 (25G9 VL), SEQ ID NO:297 (35A7-1 VL), SEQ ID NO:299 (31B9 VL), SEQ ID NO:301 (34A2 VL), SEQ ID NO:303 (34H11 VL), SEQ ID NO:305 (35D5 VL), SEQ ID NO:307 (35A7-2 VL), SEQ ID NO:309 (36F4 VL), SEQ ID NO:318 (VL1011), SEQ ID NO:319 (VL012), SEQ ID NO:320 (VL1013), or SEQ ID NO:321 (VL1014). In some embodiments, the anti-IL-4Rα antibody comprises an HCVR/LCVR sequence pair selected from the group consisting of 1A6 VH/1A6 VL; 1 D8 VH/1 D8 VL; 1H9 VH/1H9 VL; 2H1 VH/2H1 VL; 2F8 VH/2F8 VL; 9B4 VH/9B4 VL; 9E7 VH/9E7 VL; 24G10 VH/24G10 VL; 25D6 VH/25D6 VL; 25G9 VH/25G9 VL; 35A7-1 VH/35A7-1 VL; 31B9 VH/31B9 VL; 34A2 VH/34A2 VL; 34H11 VH/34H11 VL; 35D5 VH/35D5 VL; 35A7-2 VH/35A7-2 VL; and 36F4 VH/36F4 VL.

In some embodiments, an anti-IL-4Rα antibody comprises an amino acid sequence pair selected from the group consisting of: SEQ ID NOs:322/323 (13E5 VH/13E5 VL), SEQ ID NOs:324/325 (13E5H1 VH/13E5 L1 VL); and SEQ ID NOS:326/327 (13E5H4 VH/13E5 L4 VL).

In some embodiments, an anti-IL-4Rα antibody comprises an amino acid sequence pair selected from the group consisting of: SEQ ID NOs:328/329 (PD2-31 VH/PD2-31 VL) and SEQ ID NOs:330/331 (HZD82-12 VH/HZD82-12 VL).

In some embodiments, an anti-IL-4Rα antibody comprises an amino acid sequence pair selected from the group consisting of: SEQ ID NOs:332/333 (25G7 VH/25G7 VL), SEQ ID NOs:334/335 (hu25G7-VH/hu25G7-A VL), and SEQ ID NOs: 334/336 (hu25G7-VH/hu25G7-B VL).

In some embodiments, an anti-IL-4Rα antibody comprises an amino acid sequence pair selected from the group consisting of: SEQ ID NOs:337/338 (ab 136 VH/ab 136 VL); and SEQ ID NOs:339/340 (136-Hu VH/136-Hu VL).

In some embodiments, an anti-IL-4Rα antibody comprises (i) an HCVR comprising the amino acid sequence of SEQ ID NO:341 (S1E6 VH); and (ii) an LCVR comprising the amino acid sequence of SEQ ID NO:342 (L18D7 VL), SEQ ID NO:343 (L28G5 VL), SEQ ID NO:344 (L28F8 VL), SEQ ID NO:345 (L28C9 VL), SEQ ID NO:346 (101B2 VL), or SEQ ID NO:347 (L10C2 VL).

In some embodiments, an anti-IL-4Rα antibody comprises (i) an HCVR comprising the amino acid sequence of SEQ ID NO:348 (4-2 VH) or SEQ ID NO:350 (4-2-Humanized VH); and (ii) an LCVR comprising the amino acid sequence of SEQ ID NO:349 (4-2 VL) or SEQ ID NO:351 (4-2-Humanized VL). In some embodiments, an anti-IL-4Rα antibody comprises an amino acid sequence pair selected from the group consisting of: SEQ ID NOs:348/349 and SEQ ID NOs:350/351.

In some embodiments, an anti-IL-4Rα antibody comprises the amino acid sequence pair of SEQ ID NOs:352/353 (BA167 VH/BA167 VL).

In some embodiments, an anti-IL-4Rα antibody comprises the amino acid sequence pair of SEQ ID NOs:354/355 (huC2C1A1A1 VH/huB8G11F2B7G5E8 VL), SEQ ID NOs:356/355 (huB8G11 F2B7G5E8-V11 VH/huB8G11 F2B7G5E8 VL), SEQ ID NOs:357/355 (huB8G11 F2B7G5E8-V14 VH/huB8G11 F2B7G5E8 VL), SEQ ID NOs:358/359 (mB8D10G7G6E4 VH/mB8D10G7G6E4 VL), SEQ ID NOs:360/361 (mB9A7C9A4H5 VH/mB9A7C9A4H5 VL), SEQ ID NOs:362/363 (mB9D1 D11 F8D8 VH/mB9D1 D11 F8D8 VL), or SEQ ID NOs:364/365 (mB1 D2F7D3B5 VH/mB1 D2F7D3B5 VL).

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2 and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, the AbM (or “Martin”) definition; and the IMGT definition. In general terms, the Kabat definition is based on sequence variability; the Chothia definition is based on the location of the structural loop regions; the AbM definition is a compromise between the Kabat and Chothia approaches; and the IMGT definition is based on amino acid sequence alignment of germ-line V genes. See, e.g., Kabat, “Sequences of Proteins of Immunological Interest,” National Institutes of Health, Bethesda, Md. (1991); Al-Lazikani, et al., J. Mol. Biol., 273:927-948 (1997); Martin, et al., Proc. Natl. Acad. Sci. USA, 86:9268-9272 (1989); and Lefranc et al., Dev. Comp. Immunol., 27:55-77 (2003). Public databases are also available for identifying CDR sequences within an antibody.

### Pharmaceutical Compositions

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti, et al., 1991, Pharmaceut. Res., 8:1351). Specific exemplary doses of anti-IL4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science, 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem., 262:4429-4432).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348.

An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

In some embodiments, pharmaceutical compositions for use as described herein are prepared into dosage forms in a unit dose suited to fit a dose of the active ingredients. Such dosage forms in a unit dose include, for example, a vial or a prefilled syringe.

Exemplary pharmaceutical compositions comprising an anti-IL-4R antibody that can be used in the context of the present disclosure are disclosed, e.g., in U.S. Pat. No. 8,945,559.

### Dosage and Administration

In some embodiments, an IL-4R antagonist (e.g., anti-IL-4R antibody) is administered to a subject (e.g., a subject having BP) according to the methods of the present disclosure in a therapeutically effective amount. As used herein with reference to an IL-4R antagonist, the phrase “therapeutically effective amount” means an amount of IL-4R antagonist that results in one or more of: (a) an improvement in one or more symptoms of BP, such as but not limited to an improvement in the number of blisters and urticarial/eczematous lesions or an improvement in pruritus; (b) an improvement in one or more BP-associated scales or patient reported outcomes (PROs), such as but not limited to BPDAI activity score, BPDAI Pruritus score, Pruritus NRS, Skin Pain NRS, Sleep NRS, PGADS, PGAT, EQ-5D-3L, or ABQOL; (c) a decrease in the need for rescue therapy or a decrease in the amount of rescue therapy administered (e.g., the amount of OCS administered); (d) achievement of complete remission while not requiring OCS; and/or (e) an improvement in one or more BP-associated biomarkers, such as but not limited to anti-BP180 autoantibodies, anti-BP230 autoantibodies, total IgE, TARC/CCL17, and PARC/CCL18.

In the case of an anti-IL-4R antibody, a therapeutically effective amount can be from about 0.05 mg to about 800 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg of the anti-IL-4R antibody. In some embodiments, a therapeutically effective amount is from about 50 mg to about 800 mg, or from about 100 mg to about 800 mg, or from about 100 mg to about 600 mg, or from about 200 mg to about 600 mg. In certain embodiments, 50 mg, 75 mg, 100 mg, 125 mg, 150 mg, 200 mg, 250 mg, 300 mg, 350 mg, 400 mg, 450 mg, 500 mg, 550 mg, 600 mg, 650 mg, 700 mg, 750 mg, or 800 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the methods disclosed herein comprise administering an IL-4R antagonist to a subject once every week, once every two weeks, once every three weeks, or once every four weeks. In some embodiments, the methods disclosed herein comprise administering an IL-4R antagonist to a subject once a month or twice a month. In some embodiments, the methods disclosed herein comprise administering an IL-4R antagonist to a subject once every two weeks.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In some embodiments, the initial or loading dose and the one or more secondary or maintenance doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the patient 1 week, 2 weeks, 3 weeks, or 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, one or more loading doses of about 400 mg to about 600 mg of the IL-4R antagonist may be administered followed by one or more maintenance doses of about 75 mg to about 300 mg of the IL-4R antagonist. In some embodiments, an initial dose or loading dose of about 400 mg or about 600 mg of an IL-4R antagonist is administered. In some embodiments, one or more secondary doses or maintenance doses of about 200 mg or about 300 mg of the IL-4R antagonist is administered.

In some embodiments, a therapeutically effective amount of an IL-4R antagonist (e.g., anti-IL-4R antibody) comprises 300 mg administered every two weeks (Q2W). In some embodiments, a therapeutically effective amount of an IL-4R antagonist (e.g., anti-IL-4R antibody) comprises a loading dose of 600 mg followed by one or more subsequent doses of 300 mg administered every two weeks (Q2W). In some embodiments, the subject is administered a loading dose of 600 mg followed by one or more subsequent doses of 300 mg administered every two weeks (Q2W), if the subject is ≥60 kg in weight. In some embodiments, no loading dose is administered. In some embodiments, the subject is administered one or more subsequent doses of 300 mg every two weeks (Q2W), if the subject is ≥60 kg in weight.

In some embodiments, a therapeutically effective amount of an IL-4R antagonist (e.g., anti-IL-4R antibody) comprises 200 mg administered every two weeks (Q2W). In some embodiments, a therapeutically effective amount of an IL-4R antagonist (e.g., anti-IL-4R antibody) comprises a loading dose of 400 mg followed by one or more subsequent doses of 200 mg administered every two weeks (Q2W). In some embodiments, the subject is administered a loading dose of 400 mg followed by one or more subsequent doses of 200 mg administered every two weeks (Q2W), if the subject is <60 kg in weight. In some embodiments, no loading dose is administered. In some embodiments, the subject is administered one or more doses of 200 mg every two weeks (Q2W), if the subject is <60 kg in weight.

In some embodiments, a therapeutically effective amount of an IL-4R antagonist (e.g., anti-IL-4R antibody) comprises 300 mg administered every four weeks (Q4W). In some embodiments, a therapeutically effective amount of an IL-4R antagonist (e.g., anti-IL-4R antibody) comprises a loading dose of 600 mg followed by one or more subsequent doses of 300 mg administered every four weeks (Q4W). In some embodiments, a therapeutically effective amount of an IL-4R antagonist (e.g., anti-IL-4R antibody) comprises a split loading dose of 600 mg (e.g., in which 300 mg is administered on Day 1 and 300 mg is administered on Day 15) followed by one or more subsequent doses of 300 mg administered Q4W starting four weeks after the Day 15 dose. In some embodiments, the subject is administered a loading dose of 600 mg followed by one or more subsequent doses of 300 mg administered every four weeks (Q4W), if the subject is <60 kg in weight, ≥15 kg to <60 kg in weight, 30 kg in weight, or ≥15 kg to <30 kg in weight. In some embodiments, no loading dose is administered. In some embodiments, the subject is administered one or more doses of 300 mg every four weeks (Q4W), if the subject is <60 kg in weight, ≥15 kg to <60 kg in weight, <30 kg in weight, or ≥15 kg to <30 kg in weight.

In some embodiments, a therapeutically effective amount of an IL-4R antagonist (e.g., anti-IL-4R antibody) comprises 200 mg administered every four weeks (Q4W). In some embodiments, a therapeutically effective amount of an IL-4R antagonist (e.g., anti-IL-4R antibody) comprises a loading dose of 400 mg followed by one or more subsequent doses of 200 mg administered every four weeks (Q4W). In some embodiments, a therapeutically effective amount of an IL-4R antagonist (e.g., anti-IL-4R antibody) comprises a split loading dose of 400 mg (e.g., in which 200 mg is administered on Day 1 and 200 mg is administered on Day 15) followed by one or more subsequent doses of 200 mg administered Q4W starting four weeks after the Day 15 dose. In some embodiments, the subject is administered a loading dose of 400 mg followed by one or more subsequent doses of 200 mg administered every four weeks (Q4W), if the subject is <15 kg in weight or ≥5 kg to <15 kg in weight. In some embodiments, no loading dose is administered. In some embodiments, the subject is administered one or more doses of 200 mg administered every four weeks (Q4W), if the subject is <15 kg in weight or ≥5 kg to <15 kg in weight.

### Therapeutic Dosage Forms

In another aspect, the present disclosure provides therapeutic dosage forms of an IL-4R antagonist (e.g., an anti-IL-4R antibody or antigen-binding fragment thereof) for use in treating a subject having BP as disclosed herein. In some embodiments, the IL-4R antagonist is an anti-IL-4R antibody or an antigen-binding fragment thereof having one or more CDR, HCVR, and/or LCVR sequences listed in Table 1. In some embodiments, the IL-4R antagonist is an anti-IL-4Rα antibody or antigen-binding fragment thereof that comprises the HCDRs of an HCVR comprising the amino acid sequence of SEQ ID NO:1 and the LCDRs of an LCVR comprising the amino acid sequence of SEQ ID NO:2 (e.g., dupilumab).

In some embodiments, the therapeutic dose is 300 mg of the IL-4R antagonist (e.g., anti-IL-4R antibody) and is for administration every two weeks (Q2W). In the therapeutic dose is 300 mg of the IL-4R antagonist (e.g., anti-IL-4R antibody) and is for administration Q2W following a loading dose of 600 mg.

In some embodiments, the therapeutic dose is 300 mg of the IL-4R antagonist (e.g., anti-IL-4R antibody) and is for administration every four weeks (Q4W). In the therapeutic dose is 300 mg of the IL-4R antagonist (e.g., anti-IL-4R antibody) and is for administration Q4W following a loading dose of 600 mg.

In some embodiments, the therapeutic dose is 200 mg of the IL-4R antagonist (e.g., anti-IL-4R antibody) and is for administration every two weeks (Q2W). In the therapeutic dose is 200 mg of the IL-4R antagonist (e.g., anti-IL-4R antibody) and is for administration Q2W following a loading dose of 400 mg.

In some embodiments, some embodiments, the therapeutic dose is 200 mg of the IL-4R antagonist (e.g., anti-IL-4R antibody) and is for administration every four weeks (Q4W). In the therapeutic dose is 200 mg of the IL-4R antagonist (e.g., anti-IL-4R antibody) and is for administration Q4W following a loading dose of 400 mg.

### Combination Therapies

In some embodiments, the methods of the present disclosure comprise administering to the subject (e.g., a subject having BP) an IL-4R antagonist according to the disclosure (e.g., an anti-IL-4R antibody) in combination with one or more additional therapeutic agents. In some embodiments, the additional therapeutic agent is an oral corticosteroid, e.g., prednisone or prednisolone. In some embodiments, the additional therapeutic agent is a topical therapeutic agent, e.g., a TCS or a topical nonsteroidal medication such as a TCI or crisaborole. In some embodiments, the additional therapeutic agent is a systemic agent, e.g., cyclosporine A, methotrexate, mycophenolate mofetil, azathioprine, systemic or oral corticosteroid, a Janus kinase (JAK) inhibitor, or interferon-gamma. In some embodiments, the additional therapeutic agent is an immunobiologic such as a tumor necrosis factor alpha (TNFα) inhibitor (e.g., an anti-TNFα antibody such as infliximab), a CD11a inhibitor (e.g., an anti-CD11a antibody such as efalizumab), an IgE inhibitor (e.g., omalizumab), or a CD20 inhibitor (e.g., rituximab). As used herein, the expression “in combination with” means that the additional therapeutic agent is administered before, after, or concurrent with the IL-4R inhibitor. The term “in combination with” also includes sequential or concomitant administration of IL-4R inhibitor and the additional therapeutic agent.

For example, when administered “before” the pharmaceutical composition comprising the IL-4R antagonist, the additional therapeutic agent may be administered within about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered within about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than about 10 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the subject is on a background therapy. In some embodiments, the background therapy comprises an oral corticosteroid (e.g., prednisone or prednisolone). In some embodiments, the subject is on a background therapy comprising prednisone or prednisolone at a dose of about 0.5 mg/kg/day. In some embodiments, the subject is on a background therapy comprising prednisone or prednisolone at a dose of about 0.5 mg/kg/day. In certain embodiments, treatment with the IL-4R antagonist as disclosed herein results in reduced need of the background therapy. For example, in certain embodiments, treatment with the IL-4R antagonist results in a reduction in the dose of the background therapy that is administered to the subject (e.g., a reduction in daily dose of oral corticosteroid). In certain embodiments, treatment with the IL-4R antagonist results in a reduction in the frequency of the background therapy (e.g., reduction from daily dosing to less than daily dosing, e.g., 6 times a week, 5 times a week, 4 times a week, 3 times a week, 2 times a week, once a week, or less frequently).

TABLE 1
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| | LGS | Dupilumab LCDR2 amino |
| | | acid sequence |
| 8 | MQALQTPYT | Dupilumab LCDR3 amino |
| | | acid sequence |
| 9 | EVLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLG | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |
| 244 | EVLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVLVESGGGLVKPGGSLRLSCAASGFTFSMYGMHWVRQAPGKGLEWVGHIRSKS | HV3-15-14 VH |
| | SNYATYYADSVKDRFTISRDDSKNTLYLQMNSLKTEDTAVYYCTTWFRAMDYWGQ | |
| | GTLVTVSS | |
| 254 | EVLVESGGGLVQPGGSLRLSCAASGFTFSMYGMHWVRQAPGKGLEWVSHIRSKS | HV3-48-14 VH |
| | SNYATYYADSVKDRFTISRDNAKNSLYLQMNSLRAEDTAVYYCARWFRAMDYWGQ | |
| | GTLVTVSS | |
| 255 | EVLVESGGGLVQPGGSLKLSCAASGFTFSMYGMHWVRQASGKGLEWVGHIRSKS | HV3-73*2-14 VH |
| | SNYATYYADSVKDRFTISRDDSKNTAYLQMNSLKTEDTAVYYCTRWFRAMDYWGQ | |
| | GTLVTVSS | |
| 256 | EVLVESGGGLVQPGGSLRLSCAASGFTFSMYGMHWVRQAPGKGLEWVGHIRSKS | HV3-72-14 VH |
| | SNYATYYADSVKDRFTISRDDSKNSLYLQMNSLKTEDTAVYYCARWFRAMDYWGQ | |
| | GTLVTVSS | |
| 257 | EVLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27-14 VL |
| | GVPSRFSGSGSGTDFTLTISSLPEDVATYYCQQHYSTPLTFGGGTKVEIK | |
| 273 | DIQLTQSPSFLSASVGDRVTITCKASQDVSTAVAWYQQKPGKAPKLLIYWASTRHTG | KV1-9-14 VL |
| | VPSRFSGSGSGTEFTLTISSLPEDFATYYCQQHYSTPLTFGGGTKVEIK | |
| 274 | DIQMTQSPSSLSASVGDRVTITCKASQDVSTAVAWYQQKPGKAPKLLLYWASTRHT | KV1-NL1-14 VL |
| | GVPSRFSGSGSGTDYTLTISSLPEDFATYYCQQHYSTPLTFGGGTKVEIK | |
| 275 | AIRMTQSPFSLSASVGDRVTITCKASQDVSTAVAWYQQKPAKAPKLFIYWASTRHTG | KV1D-43-14 VL |
| | VPSRFSGSGSGTDYTLTISSLQPEDFATYYCQQHYSTPLTFGGGTKVEIK | |
| 276 | QVQLQQSGTELVKPGASVKMSCKASVNTFTGYNMHWIKQTPGQGLEWIG | 1A6 VH |
| | GLHPGNGDSSYNQKFKGRATLTDKSSNTAYMQLSSLTSEDSAVYYCAL | |
| | TTAGRAWFPYWGQGTLVTVSA | |
| 277 | DIQMTQTTSSLSASLGDRVTISCRASQDISNYLNWYQQKPDGTVKLLIYYTSRLHSGV | 1A6 VL |
| | PSRFSGSGSGTDYSLTISNLEQEDIATYFCQQGNTLPPTFGGGTKLEIK | |
| 278 | QVQLQQSGTELVKPGASVKMSCKASVNTFTGYNMHWIKQTPGQGLEWIG | 1D8 VH |
| | GLHPGNGDSSYNQKFKGRATLTADKSSNTAYMQLSSLTSEDSAVYYCALTTAGRAW | |
| | FAYWGQGTLVTVSA | |
| 279 | DIQMTQTTSSLSASLGDRVTISCRASQDISNYLNWYQQKPDGTVKLLIYYTSRLHSGV | 1D8 VL |
| | PSRFSGSGSGTDYSLTISNLEQEDIATYFCQQGNTLHTFGGGTKLEIK | |
| 280 | QVQLQQSGAELVKPGASVKMSCKASVNTFAGYNMHWVKQTPGQGLEWIGGLHP | 1H9 VH |
| | GNGDTSYNQKFKGKATLTADKSSNTAYMQLSSLTSEDSAVYYCALTTAGRAWFAYW | |
| | GQGTLVTVSA | |
| 281 | DIQMIQSTSSLSASLGDRVTISCRASQDISNYLNWYQQKPDGTVKLLIYYTSRLHSGVP | 1H9 VL |
| | SRFSGSGSGTDYSLTISNLEQEDIATYFCQQGNTLPWTFGGGTKLEIK | |
| 282 | QVQLQQSGAELVKPGASVKMSCKASVNTFTGYNMHWVKQTPGQGLEWIGGLHP | 2H1 VH |
| | GNGDTSYNQKFKGKATLTADKSSNTAYMQLSSLTSEDSAVYYCALTTAGRAWFAYW | |
| | GQGTLVTVSA | |
| 283 | DIQMIQSTSSLSASLGDRVTISCRASQDISNYLNWYQQKPDGTVKLLIYYTSRLHSGVP | 2H1 VL |
| | SRFSGSGSGTDYSLTISNLEQEDIATYFCQQGNTLPLTFGAGTKLELK | |
| 284 | QVQLQQSGAELVKPGASVKMSCKASVNTFTGYNMHWVKQTPGQGLEWIGGLHP | 2F8 VH |
| | GNGDTSYNQKFKGKATLTADKSSNTAYMQLSSLTSEDSAVYYCALTTAGRAWFPYW | |
| | GQGTLVTVSA | |
| 285 | DIQMIQSTSSLSASLGDRVTISCRASQDISNYLNWYQQKPDGTVKLLIYYTSRLHSGVP | 2F8 VL |
| | SRFSGSGSGTDYSLTISNLEQEDIATYFCQQGNTLPWTFGGGTKLELK | |
| 286 | QVQLQQSGAELVKPGASVKMSCKASVNTFTGYNMHWIKQTPGQGLEWIGGLHPG | 9B4 VH |
| | NGDTSYNQKFKGKATLTADKSSNTAYMQLSSLTSEDSAVYYCALTTAGRAWFAYWG | |
| | QGTLVTVSA | |
| 287 | DIQMIQSTSSLSASLGDRVTISCRASQDISNYLNWYQQKPDGTVKLLIYYTSRLHSGVP | 9B4 VL |
| | SRFSGSGSGTDYSLTISNLEQEDIATYFCQQGNTLPWTFGGGTKLELK | |
| 288 | EVQLQQSGAELVKPGASVKMSCKASVNIFTGYNMHWVKQTPGQGLEWIGGLHPG | 9E7 VH |
| | NGDTSYNQKFKDKATLTADKSSNTAYMQLSSLTSEDSAVYYCALTTAGRAWFAYWG | |
| | QGTLVTVSA | |
| 289 | DIQMTQTTSSLSASLGDRVTISCRASQDISNYLNWYQQKPDGTVKLLIYYTSRLHSGV | 9E7 VL |
| | PSRFSGSGSGTDYSLTISNLEQEDIATYFCQQGNTLPWTFGGGTKLELK | |
| 290 | EVKLVESGGGLVKPGGSLKLSCAASGFTFSRYAMSWVRQTPEKRLEWVATISSGGSY | 24G10 VH |
| | TNYADSVKGRFTISRDNVKNTLYLQMNSLRAEDTAVYYCARATARATEFAYWGQGT | |
| | LVTVSS | |
| 291 | DIQMTQTTSSLSASLGDRVTISCRASQDISNYLNWYQQKPDGTVKLLIY | 24G10 VL |
| | YTSRLHSGVPSRFSGSGSGTDYSLTISNLEQEDIATYFCQQGNTLPPTFGGGTKLEL | |
| 292 | EVQLQQSGAELVKPGASVKMSCKASVNIFTGYNMHWVKQTPGQGLEWIGGLHPG | 25D6 VH |
| | NGDTSYNQKFKDKATLTADKSSNTAYMQLSSLTSEDSAVYYCALTTAGRAWFAYWG | |
| | QGTLVTVSA | |
| 293 | DIQMTQSTSSLSASLGDRVTISCRASQDISNYLNWYQQKPDGTVKLLIYYTSRLHSGV | 25D6 VL |
| | PSRFSGSGSGTDYSLTISNLEQEDIATYFCQQGNTLPWTFGGGTKLEIK | |
| 294 | EVQLKESGAELVKPGASVKMSCKASVNIFTGYNMHWVKQTPGQGLEWIGGLHPG | 25G9 VH |
| | NGDTSYNQKFKDKATLTADRSSNTAYMQLSSLTSEDSAVYYCALTTAGRAWFAYWG | |
| | QGTLVTVSA | |
| 295 | DIQMTQSTSSLSASLGDRVTISCRASQDISNYLNWYQQKPDGTVKLLIYYTSRLHSGV | 25G9 VL |
| | PSRFSGSGSGTDYSLTISNLEQEDIATYFCQQGNTLPWTFGGGTKLEIK | |
| 296 | QVQLKQSGAELVKPGASVKMSCTASVNIFTGYNMHWIKQTPGQGLEWIG | 35A7-1 VH |
| | GLHPGNGDTSYNQKFKDKATLTADRSSNTAYMQLSSLTSEDSAVYYCAL | |
| | TTAGRAWFAYWGQGTLVTVSA | |
| 297 | DIQMNQSTSSLSASLGDRVTISCRASQDISNYLNWYQQKPDGTIKLLIYYTSRLYSGVP | 35A7-1 VL |
| | SRFSGSGSGTDYSLTISNLEEEDIATYFCQQGNTIPYTFGGGTKLEIK | |
| 298 | EVQLQQSGAELVKPGASVKMSCKASVNIFTGYNMHWVKQTPGQGLEWIGGLHPG | 31B9 VH |
| | NGDTSYNQKFKDKATLTADRSSNTAYMQLSSLTSEDSAVYYCALTTAGRAWFAYWG | |
| | QGTLVTVSA | |
| 299 | DIQMTQSTSSLSASLGDRVTISCRASQDISNYLNWYQQKPDGTIKLLIYYTSRLHSGVP | 31B9 VL |
| | SRFSGSGSGTDYSLTISNLEEEDIATYFCQQGNTLPWTFGGGTKLEIK | |
| 300 | EVQLQQSGAELVKPGASVKMSCKASVNIFTSYNMHWVKQTPGQGLEWIGGLHPG | 34A2 VH |
| | NGDTSYNQKFKGKATLTADKSSSTAYMQVSMLTSEDSAVYYCVLTTAGRAWFAYW | |
| | GQGTLVTVSA | |
| 301 | DIQMTQSTSSLSASLGDRVTISCRASQDISNYLNWYQQKPDGTIKLLIYYTSRLHSGVP | 34A2 VL |
| | SRFSGSGSGTDYSLTISNLEEEDIATYFCQQGNTLPWTFGGGTKLEIK | |
| 302 | QVQLQESGAELVKPGASVKMSCKASVNTFTGYNMHWVKQTPGQGLEWIGGLHPG | 34H11 VH |
| | NGDTSYNQKFKGKATLTADRSSNTAYMQLSSLTSEDSAVYYCALTTAGRAWFAYWG | |
| | QGTLVTVSA | |
| 303 | DIQMTQTTSSLSASLGDRVTISCRASQDISNYLNWYQQKPDGTVKLLIYYTSRLHSGV | 34H11 VL |
| | PSRFSGSGSGTDYSLTISNLEEEDIATYFCQQGNTLPWTFGGGTKLEIK | |
| 304 | QVQLKQSGAELVKPGASVKMSCKASVNTFTGYNMHWVKQTPGQGLEWIGGLHPG | 35D5 VH |
| | NGDTSYNQKFKGKATLTADKSSNTAYMQLSSLTSEDSAVYYCALTTAGRAWFAYWG | |
| | QGTLVTVSA | |
| 305 | DIQMTQSTSSLSASLGDRVTISCRASQDISNYLNWYQQKPDGTVKLLIYYTSRLHSGV | 35D5 VL |
| | PSRFSGSGSGTDYSLTISNLEEEDIATYFCQQGNTLPWTFGGGTKLEIK | |
| 306 | QVQLKQSGAELVKPGASVKMSCKASVNTFTGYNMHWIKQTPGQGLEWIGGLHPG | 35A7-2 VH |
| | NGDTSYNQKFKGKATLTADKSSNTAYMQLSSLTSEDSAVYYCALTTAGRAWFAYWG | |
| | QGTLVTVSA | |
| 307 | DIQMNQSTSSLSASLGDRVTISCRASQDISNYLNWYQQKPDGTIKLLIYYTSRLYSGVP | 35A7-2 VL |
| | SRFSGSGSGTDYSLTISNLEEEDIATYFCQQGNTLPYTFGGGTKLEIK | |
| 308 | QVQLQQSGAELVKPGASVKMSCKASVNTITGYNMHWVKQTPGQGLEWIGGLHPG | 36F4 VH |
| | NGDTSYNQKFKDKATLTADKSSNTAYMQLSSLTSEDSAVYYCALTTAGRAWFAYWG | |
| | QGTLVTVSA | |
| 309 | DIKMTQSTSSLSASLGDRVTTSCRASQDISNYLNWYQQKPDGTVKLLIYYTSRLHSGV | 36F4 VL |
| | PSRFSGSGSGTDYSLTISNLEEEDIATYFCQQGNTLPPWTFGGGTKLEIK | |
| 310 | EVLVESGGGLVKPGGSLRLSCAASGFTFSRYAMSWVRQAPGKGLEWVS | VH1021 |
| | TISSGGSYTNYADSVKGRFTISRDNVKNTLYLQMNSLRAEDTAVYYCARATARATEFA | |
| | YWGQGTLVTVSS | |
| 311 | EVQLLESGGGLVQPGGSLRLSCAASGFTFSRYAMSWVRQAPGKGLEWVS | VH1022 |
| | TISSGGSYTNYADSVKGRFTISRDNVKNTLYLQMNSLRAEDTAVYYCARATARATEFA | |
| | YWGQGTLVTVSS | |
| 312 | QVQLVQSGAELKKPGASVKVSCKASGNTFTGYNMHWIQQSPGQGLEWMGGLHP | VH1023 |
| | GNGDSSYNQKFQGRVTLTADKSSNTAYMELSSLRSEDSAVYYCALTTAGRAWFPYW | |
| | GQGTLVTVSS | |
| 313 | QVQLVQSGAEVKKPGASVKMSCKASVYTFTGYNMHWIQQSPGQGLEWMGGLHP | VH1024 |
| | GNGDSSYNQKFQGRATLTADKSSNTAYMELSSLRSEDSAVYYCALTTAGRAWFPYW | |
| | GQGTLVTVSS | |
| 314 | QVQLVQSGAEVKKPGASVKVSCKASGNIFTGYNMHWVRQAPGQGLEWMGGLHP | VH1025 |
| | GNGDSSYNQKFQGRVTLTADKSSNTAYMELSSLRSEDSAVYYCALTTAGRAWFAYW | |
| | GQGTLVTVSS | |
| 315 | QVQLVQSGAEVKKPGASVKMSCKASVYTFTGYNMHWVRQAPGQGLEWIGGLHP | VH1026 |
| | GNGDSSYNQKFQGRATLTADKSSNTAYMELSSLRSEDSAVYYCALTTAGRAWFAYW | |
| | GQGTLVTVSS | |
| 316 | EVQLVQSGAEVKKPGATVKISCKASGNIFTSYNMHWVRQAPGQGLEWMGGLHPG | VH1027 |
| | NGDTSYNQKFQGRVTLTADKSSSTAYMELSSLRSEDTAVYYCVLTTAGRAWFAYWG | |
| | QGTLVTVSS | |
| 317 | EVQLVQSGAEVKKPGATVKMSCKASVYTFTSYNMHWVRQAPGQGLEWIGGLHPG | VH1028 |
| | NGDTSYNQKFKGRATLTADKSSSTAYMELSSLRSEDTAVYYCVLTTAGRAWFAYWG | |
| | QGTLVTVSS | |
| 318 | DIQMTQSPSSLSASVGDRVTITCQASQDISNYLNWYQQKPGKAPKLLIYYTSRLHSGV | VL1011 |
| | PSRFSGSGSGTDYTFTISSLQPEDIATYFCQQGNTLPWTFGGGTKVEIK | |
| 319 | DIQMTQSPSSLSASVGDRVTITCRASQDISNYLNWYQQKPGKAPKLLIYYTSRLHSGV | VL1012 |
| | PSRFSGSGSGTDYTLTISSLQPEDFATYFCQQGNTLPWTFGGGTKVEIK | |
| 320 | DIQMTQSPSSLSASVGDRVTITCQASQDISNYLNWYQQKPGKAPKLLIYYTSRLHSGV | VL1013 |
| | PSRFSGSGSGTDYTLTISSLQPEDFATYFCQQGNTLPLTFGGGTKVEIK | |
| 321 | DIQMTQSPSSLSASVGDRVTITCQASQDISNYLNWYQQKPGKAPKLLIYYTSRLHSGV | VL1014 |
| | PSRFSGSGSGTDYTLTISSLQPEDFATYFCQQGNTLPWTFGGGTKVEIK | |
| 322 | EVQLQQSGPELVKPGASVKISCKTSGYTFTEYTIHWVKQNHGKSLEWIGGINPNNGG | 13E5 VH |
| | TVYNQNFKGKATLVDKSSSTAYMELRSLTSEDSAVYYCARVRRGMDYWGQGTSVTV | |
| | SS | |
| 323 | DIVMTQSHKFMSTSVGDRVSITCKASQDVTTAVAWYQQKPGQSPKLLIYSASYRYTG | 13E5 VL |
| | VPDRFTGSGSGTDFTFTISSVQAEDLAVYYCQQHYSAPWTFGGGTNLEIK | |
| 324 | QVQLQQSGAEVVKPGASVKISCKTSGYTFTEYTIHWVKQAHGQSLEWIGGINPNNG | 13E5 H1 VH |
| | GTVYNQKFQGKATLTVDKSTSTAYMELRSLTSEDTAVYYCARVRRGMDYWGQGTS | |
| | VTVSS | |
| 325 | DIQMTQSPKSTSTSVGDRVTITCRASQDVTTAVAWYQQKPGKSPKLLIYSASYRYTG | 13E5 L1 VL |
| | VPSRFSGSGSGTDFTFTISSVQPEDLATYYCQQHYSAPWTFGGGTNLEIK | |
| 326 | QVQLVQSGAEVVKPGASVKVSCKTSGYTFTEYTIHWVRQAPGQSLEWIGGINPNNG | 13E5 H4 VH |
| | GTVYNQKFQGKVTLTVDKSTSTAYMELSSLRSEDTAVYYCARVRRGMDYWGQGTS | |
| | VTVSS | |
| 327 | DIQMTQSPSSLSASVGDRVTITCRASQDVTTAVAWYQQKPGKAPKLLIYSASYRYTG | 13E5 L4 VL |
| | VPSRFSGSGSGTDFTLTISSVQPEDLATYYCQQHYSAPWTFGGGTNLEIK | |
| 328 | EVLVESGGGLVQPGGSLRLSCAASGIDLRTNSMSWVRQAPGKGLEWVGIIGSSGY | PD2-31 VH |
| | MDYASWAKGRFTISRDNSKNTLYLQMNSLRAEDTAVYYCARHGDSSSFALWGQGT | |
| | LVTVSS | |
| 329 | DIQMTQSPSSLSASVGDRVTITCRASESVYKNNRLSWYQQKPGKAPKLLIYEASKVAS | PD2-31 VL |
| | GVPSRFSGSGSGTDFTLTISSLPEDFATYYCAGAYRGNIYPFGQGTKVEIK | |
| 330 | EVLVESGGGLVQPGGSLRLSCAASGFSLNSNAVGWVRQAPGKGLEYIGFINFSGIT | HZD82-12 VH |
| | YYANWAKGRFTISRDNSKNTLYLQMNSLRAEDTAVYYCARGSAGSFDLWGQGTLVT | |
| | VSS | |
| 331 | DIQMTQSPSSLSASVGDRVTITCQASESVYKNNYLAWYQQKPGKAPKLLIYRASNLAS | HZD82-12 VL |
| | GVPSRFSGSGSGTDFTLTISSLPEDFATYYCQGGYSSGMYPFGQGTKVEIK | |
| 332 | EVLVESGGGLVKPGGSLKLSCAASGFTFSDYGMHWVRQAPEKGLEWVAFISSGSSI | 25G7 VH |
| | IYYADIVKGRSTISRDNAKNTLFLQMTSLRSEDTAMYYCTRGNKRGFFDYWGQGTILT | |
| | VSS | |
| 333 | QIVLTQSPALMSASPGEKVTMTCNASSSVSYMYWYQRKPRSSPKPWIYLTSNLASG | 25G7 VL |
| | VPVRFSGSGSGTSYSLTISSMEAEDAATYYCQQWRSNPPMLTFGSGTKLEVK | |
| 334 | EVLVESGGGLVQPGGSLRLSCAASGFTFSDYGMHWVRQAPGKGLEWVAFISSGSS | hu25G7-VH |
| | IIYYADIVKGRSTISRDNAKNTLYLQMNSLRAEDTAVYYCTRGNKRGFFDYWGQGTL | |
| | VTVSS | |
| 335 | EIVLTQSPATLSLSPGERATLSCRASSSVPYMYWYQQKPGQAPRLLIYLTSNLASGIPA | hu25G7-A VL |
| | RFSGSGSGTDFTLTISSLEPEDFAVYYCQQWRAYPPMLTFGGGTKVEIK | |
| 336 | EIVLTQSPATLSLSPGERATLSCRASPGVPPLAWYQQKPGQAPRLLIYLASSRPSGIPA | hu25G7-B VL |
| | RFSGSGSGTDFTLTISSLEPEDFAVYYCQQWRSNPPMLTFGGGTKVEIK | |
| 337 | EVLVESGGGLVKPGGSLKLSCAASGFTFSDYGMHWVRQAPEKGLEWVAYISSGST | ab 136 VH |
| | TIYYADTVKGRFTISRDNGKNTLFLQMTSLRSEDTAMYYCARISTVVAKRYAMD | |
| | YWGQGTSVTVSS | |
| 338 | DIQMTQTASSLSASLGDRVTISCRASQDISNYLNWYQQKPDGTVKLLIYYTSRLHSGV | ab 136 VL |
| | PSRFSGSGSGTDYSLTISNLEQEDIATYFCQQINALPLTFGAGTKLELK | |
| 339 | EVLVESGGGLVQPGGSLRLSCAASGFTFSDYGMHWVRQAPGKGLEWVSYISSGST | 136-Hu VH |
| | TIYYADTVKGRFTISRDNAKNSLYLQMNSLRAEDTAVYYCARISTVVAKRYAMDYWG | |
| | QGTLVTVSS | |
| 340 | DIQMTQSPSSLSASVGDRVTITCRASQDISNYLNWYQQKPGKAPKLLIYYTSRLHSGV | 136-Hu VL |
| | PSRFSGSGSGTDFTLTISSLQPEDIATYYCQQINALPFTFGQGTKLEIK | |
| 341 | EVLVESGGGLVQPGGSLRLSCAVSGFTFSSYAMSWVRQAPGKGLEWVSSITGGG | S1E6 VH |
| | GGIYYADSVKGRFTISRDNSKNTVYLQMNSLRAEDTAVYYCAKDRISITIRPRYFGLDF | |
| | WGQGTTVTVSS | |
| 342 | DIVMTQSPLSLPVTPGEPASISCRSSQSLLYSIGYNYLDWYLQKSGQSPQLLIYLGSNR | L18D7 VL |
| | ASGVPDRFSGSGSGTDFTLKISRVEAEDVGFYYCMQSFKAPYTFGQGTKLEIK | |
| 343 | DIVMTQSPLSLPVTPGEPASISCRSSRNVIYGNGYNYLDWYLQKSGQSPQLLIYLGNN | L28G5 VL |
| | VAAGVPDRFSGSGSGTDFTLKISRVEAEDVGFYYCMQSLQAPYTFGQGTKLEIK | |
| 344 | DIVMTQSPLSLPVTPGEPASISCRSSQNVVYGNGYNYLDWYLQKSGQSPQLLIYLGTN | L28F8 VL |
| | VAAGVPDRFSGSGSGTDFTLKISRVEAEDVGFYYCMQSLQAPYTFGQGTKLEIK | |
| 345 | DIVMTQSPLSLPVTPGEPASISCRSSQNVVYGNGYNYLDWYLQKSGQSPQLLIYLGN | L28C9 VL |
| | NVAAGVPDRFSGSGSGTDFTLKISRVEAEDVGFYYCMQSLKAPYTFGQGTKLEIK | |
| 346 | DIVMTQSPLSLPVTPGEPASISCRSSHNLLYSNGYNYLDWYLQKPGQSPQLLIYLGSN | L10B2 VL |
| | RAYGVPDRFSGSGSGTDFTLKISRVEAEDVGVYHCMQALQSPYTFGQGTKVDIK | |
| 347 | DIVMTQSPLSLPVTAGAPASISCRSSQSLLYSNGYNYLDWYMQKPGQSPQLLIYLGSN | L10C2 VL |
| | RASGVPDRFSGSGSGTDFTLKISRVEAEDVGVYYCMQALETPYAFGQGTKVEIK | |
| 348 | QVQLQQSGPELVKPGASVKISCTASGSTLTDDYINWVKQRPGRGLEWVGWIFPGN | 4-2 VH |
| | GNSYYNEKFKDKATLIVDKSSSTAYMLLSSLTSEDSAVYFCARGLVRYRALFDYWGQG | |
| | TTLTVSS | |
| 349 | QIVLSQSPAILSASPGEKVTMTCRASSSINYMHWYQQKPGSSPKPWIYAASNLASGV | 4-2 VL |
| | PARFSGSGSGTSFSLTISRVEAEDAATYYCQQWSSYPITFGSGTKLEIK | |
| 350 | QVQLVQSGAEVKKPGASVKVSCKASGSTLTDDYINWVRQAPGQRLEWVGWIFPGN | 4-2-Humanized VH |
| | GNSYYNEKFKDRATLTVDKSASTAYMELSSLRSEDTAVYFCARGLVRYRALFDYWGQ | |
| | GTLVTVSS | |
| 351 | DIQMTQSPSSLSASVGDRVTITCRASSSINYMHWYQQKPGKAPKPWIYAASNLASG | 4-2-Humanized VL |
| | VPSRFSGSGSGTDFTLTISSLPEDFATYYCQQWSSYPITFGQGTKVEIK | |
| 352 | QVQLVESGGGVVQPGRSLRLSCAASGFTFSSYAMHWVRQAPGKGLEWVAVISYDG | BA167 VH |
| | SNKYYADSVKGRFTISRDNSKNTLYLQMNSLRAEDTAVYYCARGLTTVRGVLYWGQ | |
| | GTLVTVSS | |
| 353 | EIVMTQSPSSLSASLGDRVTITCRASQNIGSRLAWYQQKPGKAPKLLIYKASSLESGVP | BA167 VL |
| | SRFSGSGSGTEFTLTISSLQPDDFATYYCQQYNSYSWTFGQGTKLEIK | |
| 354 | EVLVESGGGLVQPGGSLRLSCAASGFTFSTYGMSWVRQAPGKGLVXVXTINSNGG | huC2C1A1A1 VH |
| | STSYPDSVKGRFTISRDNAKNTLYLQMNSLRAEDTAVYYCARFFRFRNAMDYWGQG | |
| | TLVTVSS | |
| 355 | DIVMTQTPLSLSVTPGQPASISCRSSQSIVHSNGNTYLEWYLQKPGQSPQLLIYKVTN | huB8G11F2B7G5E8 VL |
| | RFSGVPDRFSGSGSGTDFTLKISRVEAEDVGVYYCFQGSHVPYTFGQGTKLEIK | |
| 356 | EVLVQSGAEVKKPGATVKISCKVSGFNIKDTYMHWVQQAPGKGLEWMGLIDPTN | huB8G11F2B7G5E8-V11 |
| | GYTIYAEKFQGRVTITADTSTDTAYMELSSLRSEDTAVYYCVSRRPWFAYWGQGTLV | VH |
| | TVSS | |
| 357 | QVQLQQSGAELVKPGASVKLSCTASGFNIKDTYMHWVKQRPEQGLEWVGRIDPTN | huB8G11F2B7G5E8-V14 |
| | GYTIYASKFQGKATITADTSSNTAYMQLSSLTSEDTAVYHCVSRRPWFAYWGQGTTL | VH |
| | TVSS | |
| 358 | EVLVESGGGLVKPGGSLKLSCAASGFTFSSYAMSWVRQTPEKRLEWVAGIRSGGS | mB8D10G7G6E4 VH |
| | YTYYPDTVKGRFTISRDNARNTLYLQMNSLRSEDTAIYYCARGDKLRPYHFDYWGQG | |
| | TTLTVSS | |
| 359 | DIVMTQSHKFMSTSVGDKVSITCKASQDVTTAVAWYQQKPGQSPKLLIYSASYRYTG | mB8D10G7G6E4 VL |
| | VPDRFAGSGSGTDFTVTISTVQAEDLAVYYCQQHYSDPYTFGGGTKLEIK | |
| 360 | EVLVESGGGLVKPGGSLKLSCAASGFTFSNYAMSWVRQTPEKRLEWVAGIRSGGS | mB9A7C9A4H5 VH |
| | YTYYPDTVKGRFTISRDNARNTLYLQMSSLRSEDTAIYYCARGDKLRPYHFDYWGQG | |
| | TTLTVSS | |
| 361 | DIVMTQSHKFMSTSVGDRVSITCKASQDVSTAVVWYQQKPGQSPKLLIYSASYRYTG | mB9A7C9A4H5 VL |
| | VPDRFTGSGSGTDFTFTIITVQAEDLAVYYCQQHYSAPYTFGGGTQLEIK | |
| 362 | EVKLVESGGGLVKPGGSLKLSCAASGFTFSSYAMSWVRQTPEKRLEWVASISSGDST | mB9D1D11F8D8 VH |
| | YYLDSVKGRFTISRDNARNILYLQVSSLRSEDTAMYYCERSGGSAPYWGQGTLVTVSA | |
| 363 | QIVLTQSPAIMSASPGDMVTISCSASSSVNYMYWYQQKPGSSPKPWIYRTSNLASG | mB9D1D11F8D8 VL |
| | VPARFSGSGSGTSYSLTISSMEAEDAATYYCQQYHSFPLTFGAGTKLELK | |
| 364 | EVKLVESGGGLVKPGGSLKLSCAASGFTFSSYAMSWVRQTPEKRLEWVASISSGDST | mB1D2F7D3B5 VH |
| | YYLDSVKGRFTISRDNAMNILYLQMSSLRSEDTAVYYCERSGGSAPYWGQGTLVSVS | |
| | A | |
| 365 | QIVLTQSPAIMSASPGEMVTISCSASSSVSYMYWYQQKPGSSPKPWIYRTSNLASGV | mB1D2F7D3B5 VL |
| | PARFSGSGSGTSYSLTISSMEAEDAATYYCQQYHSFPLTFGAGTKLELK | |
| 366 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | with C-terminal lysine |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial to Assess the Efficacy, Safety, Tolerability, Pharmacokinetics, and Immunogenicity of Dupilumab in Patients with Bullous Pemphigoid

This example describes a Phase 3, randomized, double-blind, placebo-controlled clinical trial to investigate the efficacy and safety of dupilumab in adult patients with moderate-to-severe bullous pemphigoid (BP).

### Study Design and Objectives

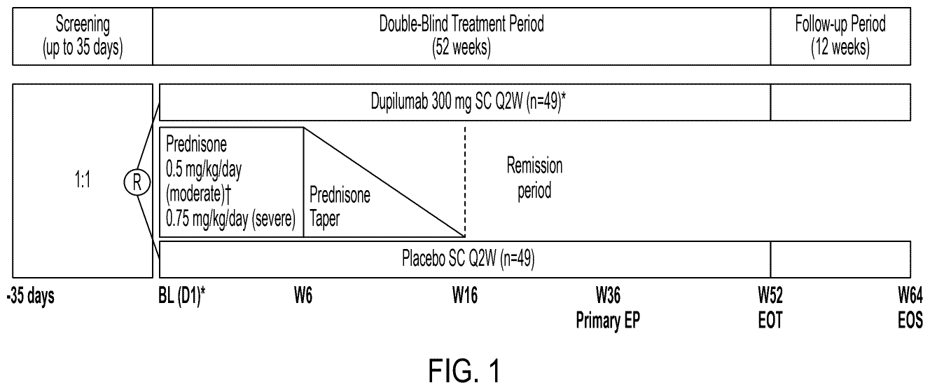

An overview of the study is shown in FIG. 1. The study included a 35-day screening period; a 52-week, double-blind treatment period; and a 12-week follow-up period. Study participants were required to have characteristic clinical features of BP present at the screening visit (e.g., urticarial, eczematous or erythematous plaques, bullae, pruritus), and a confirmed diagnosis of BP based on histopathology, immunopathology, and serology at the baseline visit. Patients were also required to have moderate-to-severe BP at the screening and baseline visits, as determined by a BPDAI activity score ≥24, and a peak pruritus NRS for maximum itch intensity ≥4 at the baseline visit.

At the baseline visit, eligible patients were randomized to receive dupilumab or matching placebo. Randomization was stratified by baseline disease severity (moderate BP vs. severe BP), region (North America vs. Europe vs. Japan), and prior corticosteroid/immunosuppressant use (Yes vs. No). Approximately 10% of the study population was planned to be from Japan; patients from study sites in Japan were not stratified. In addition, all patients received a standard regimen of OCS (prednisone or prednisolone) beginning at the baseline visit to obtain control of disease activity. Specifically, patients with moderate BP (BPDAI activity score ≥24 and <60) received 0.5 mg/kg/day of prednisone (or prednisolone) and patients with severe BP (BPDAI activity score ≥60) received 0.75 mg/kg/day of prednisone (or prednisolone). Patients were maintained on their assigned treatment level of OCS until they achieved control of disease activity. After achieving sustained control of disease activity, patients followed a protocol-defined OCS tapering regimen as long as they maintained control of disease activity. Tapering of OCS started after 2 weeks of sustained control of disease activity. Following the OCS taper period, there was a remission period of at least 36 weeks where patients were observed for relapses off OCS.

The primary objective of the study was to demonstrate that dupilumab is superior to placebo in achieving sustained remission off oral corticosteroid (OCS) in patients with BP.

The secondary objectives of the study were:
To evaluate the OCS-sparing effects of dupilumab in patients with BP
To evaluate the effect of dupilumab on itch in patients with BP
To evaluate the effects of dupilumab on health-related quality of life measures in patients with BP
To evaluate the effect of dupilumab in circulating BP180 and BP230 autoantibody titers
To assess the safety and tolerability of dupilumab administered to patients with BP
To characterize the trough concentrations of functional dupilumab over time following administration of dupilumab in patients with BP
To assess the immunogenicity of dupilumab in patients with BP over time

The exploratory objectives of the study were:
To evaluate the incidence and time to relapse following control of disease activity
To evaluate the effect of dupilumab on skin pain in patients with BP
To evaluate the effect of dupilumab on sleep in patients with BP
To evaluate the effects of dupilumab on the inflammatory profile in circulation, as measured by serum and plasma chemokines/cytokines, immunoglobulin levels, and gene expression
To evaluate the effects of dupilumab on inflammatory profiles in the peri-lesional skin of patients with BP as measured by gene expression (may include RNAscope) and immunohistological analysis of skin biopsies

### Endpoints

The primary endpoint was the proportion of patients achieving sustained remission at week 36, defined as:
achievement of complete remission and off OCS no later than week 16 after randomization, and
absence of disease relapse from the time the patient has completed the OCS taper (no later than week 16 after randomization) to week 36, and
absence of need for rescue therapy during the 36-week double-blind treatment period

The key secondary endpoints were:
total cumulative dose of OCS from baseline to week 36
percent change in weekly average of daily peak pruritus NRS from baseline of week 36
proportion of patients with reduction of weekly average of daily peak pruritus NRS 4 from baseline to week 36
percent change in BPDAI activity score from baseline to week 36
time to first use of rescue mediation (up to week 36)

Other secondary endpoints for efficacy were:
duration of complete remission while not requiring OCS (up to week 36)
proportion of patients who do not achieve control of disease activity, who relapse after achieving control of disease activity, or do not achieve complete remission (up to week 36)
proportion of patients with reduction in BPDAI activity score ≥50, ≥75, or ≥90% from baseline to week 36
change in autoimmune bullous disease quality of life (ABQOL) from baseline to week 36
change from baseline to week 36 in percent body surface area (BSA) of BP involvement
change in BP180 and BP230 autoantibody (Immunoglobulin G [IgG]) titers from baseline to week 36
proportion of patients with sustained remission at week 52
total cumulative dose of OCS from baseline to week 52
duration of complete remission while not requiring OCS (up to week 52)
proportion of patients who do not achieve control of disease activity or, who relapse after achieving control of disease activity, or do not achieve complete remission (up to week 52)
percent change in weekly average of daily peak pruritus NRS from baseline to week 52
proportion of patients with improvement (reduction) of weekly average of daily peak pruritus NRS ≥4 from baseline to week 52
percent change in BPDAI activity score from baseline to week 52
proportion of patients who achieve a reduction in BPDAI activity score of at least 50%, 75%, and 90% from baseline to week 52
change in ABQOL from baseline to week 52
change from baseline to week 52 in percent BSA of BP involvement
change in BP180 and BP230 autoantibody (IgG) titers from baseline to week 52
proportion of patients in complete remission and off OCS at week 16
percent change in BPDAI activity score from baseline to week 16
proportion of patients who achieve a reduction in BPDAI activity score of at least 50%, 75%, and 90% from baseline to week 16
percent change in weekly average of daily peak pruritus NRS from baseline to week 16
proportion of patients with improvement (reduction) of weekly average of daily peak pruritus NRS 4 from baseline to week 16

Other secondary endpoints for safety were:
incidence of treatment-emergent adverse events (TEAEs) from baseline through end of treatment (up to week 52)
incidence of treatment-emergent serious adverse events (SAEs) from baseline through the end of treatment (up to week 52)
incidence of adverse events of special interest (AESIs) from baseline through the end of treatment (up to week 52)
incidence of TEAEs from baseline through end of study (up to week 64)
incidence of treatment-emergent SAEs from baseline through the end of study (up to week 64)
incidence of AESIs from baseline through the end of study (up to week 64)

### Patient Criteria

The following were inclusion criteria for the trial:
(1) Characteristic clinical features of BP (e.g., urticarial or eczematous or erythematous plaques, bullae, pruritus) at the screening and baseline visits
(2) A confirmed diagnosis of BP based on histopathology, immunopathology, and serology at the baseline visit. Required for inclusion:
   (a) Compatible histopathologic findings on a skin biopsy specimen supporting a diagnosis of BP (e.g., a subepidermal blister with eosinophils within the cleft, a mixed cellular inflammatory infiltrate that includes eosinophils) AND
   (b) Characteristic direct immunofluorescence findings on a skin biopsy specimen demonstrating linear IgG and/or C3 at the basement membrane zone AND
   (c) At least 1 of the following serologic assessments (utilizing a peripheral blood specimen): (i) positive indirect immunofluorescence demonstrating IgG antibodies localizing to the roof (epidermal side) of split-skin substrate; (ii) increased IgG serum antibody levels by immunoassay to BP antigens, BPAG1 (230-kd, also known as BP230) and/or BPAG2 (180-kd, also known as BP180)
(3) BPDAI activity score ≥24 at baseline and screening visits
(4) Baseline peak pruritus NRS score for maximum itch intensity 4. NOTE: Baseline peak pruritus NRS score for maximum itch intensity will be determined based on the average of daily NRS scores for maximum itch intensity (the daily score ranges from 0 to 10) during the 7 days immediately preceding randomization. A minimum of 3 daily scores out of the 7 days is required to calculate the baseline average score.
(5) Male or female, age 18 to 90 at the screening visit (age 20 to 90 for sites in Japan)
(6) Karnofsky performance status score ≥50% at the screening visit
(7) Willing and able to comply with clinic visits and study-related procedures
(8) Willing and able to understand and complete study-related questionnaires
(9) Willing and able to provide voluntary signed informed consent

The following were exclusion criteria for the trial:
(1) Forms of pemphigoid other than classic BP (e.g., Brunsting-Perry cicatricial pemphigoid, anti-p200 pemphigoid, epidermolysis bullosa acquisita, or BP with concomitant pemphigus vulgaris)
(2) Patients who are receiving treatments known to cause or exacerbate BP (e.g., angiotensin converting enzyme inhibitors, penicillamine, furosemide, phenacetin, dipeptidyl peptidase 4 inhibitor) who have not been on a stable dose of these medications for at least 4 weeks prior to the screening visit
(3) Have ever received treatment with an IL-4 or IL-13 antagonist such as dupilumab, tralokinumab, or lebrikizumab
(4) Treatment with systemic corticosteroids within 7 days before the baseline visit
(5) Treatment with topical corticosteroids of medium potency or higher, topical calcineurin inhibitor, or topical crisaborole within 7 days before the baseline visit
(6) Initiation, discontinuation, or change in the dosage regimen of systemic antihistamines within 7 days before the baseline visit. Patients on a stable dose of systemic antihistamines for at least 7 days prior to the baseline visit may be included in the study; however, the dose of these medications should remain unchanged for the duration of the study.
(7) Treatment with an antibiotic (such as doxycycline or dapsone) directed at the treatment of BP within 7 days before the baseline visit
(8) Treatment with nicotinamide directed at the treatment of BP within 7 days prior to the baseline visit (note: use of multivitamins containing nicotinamide is allowed)
(9) Treatment with non-steroidal immunosuppressive/immunomodulating drug(s) (e.g., mycophenolate mofetil, azathioprine, or methotrexate) within 4 weeks before the baseline visit
(10) Treatment with BP-directed biologics as follows:
   (a) any cell-depleting agents including but not limited to rituximab: within 12 months before the baseline visit, or until lymphocyte and CD 19+ lymphocyte count returns to normal, whichever is longer
   (b) other biologics (such as IL-5 inhibitors benralizumab or mepolizumab): within 5 half-lives (if known) or 16 weeks prior to the baseline visit, whichever is longer
   (c) intravenous immunoglobulin within 16 weeks prior to the baseline visit
(11) Treatment with a live (attenuated) vaccine within 4 weeks before the baseline visit
(12) Planned or anticipated use of any prohibited medications or procedures during study treatment
(13) Any active infection requiring systemic treatment; patients with such infection must have their infection resolved at least 1 week before baseline to be eligible to enroll in the study
(14) Diagnosed active endoparasitic infections; suspected or high risk of endoparasitic infection, unless clinical and (if necessary) laboratory assessment have ruled out active infection before randomization
(15) Known or suspected history of immunosuppression, including history of invasive opportunistic infections (e.g., tuberculosis [TB], histoplasmosis, listeriosis, coccidioidomycosis, pneumocystosis, aspergillosis) despite infection resolution: or unusually frequent, recurrent, or prolonged infections, per investigator's judgment
(16) History of human immunodeficiency virus (HIV) infection or positive HIV serology at screening.
(17) A current diagnosis of hepatitis B viral infection at the time of screening as evidenced by (a) positive hepatitis B surface antigen (HBsAg) or (b) positive total hepatitis B core antibody (HBcAb) confirmed by positive hepatitis B virus (HBV) DNA
(18) A current diagnosis of hepatitis C viral infection at the time of screening as evidenced by (a) positive hepatitis C virus (HCV) Ab AND (b) positive HCV RNA
(19) On current treatment for hepatic disease including, but not limited to, acute or chronic hepatitis, cirrhosis, or hepatic failure, or has evidence of liver disease as indicated by persistent (confirmed by repeated tests ≥2 weeks apart) elevated transaminases (alanine aminotransferase [ALT] and/or aspartate aminotransferase [AST]) more than 3 times the upper limit of normal (ULN) during the screening period
(20) Presence of any 1 or more of the following abnormalities in laboratory test results at screening: (a) platelet count≤100×10³/μL; (b) neutrophil count <1.5×10³/μL
(21) Presence of skin comorbidities that may interfere with study assessments
(22) History of malignancy within 5 years before the baseline visit, except completely treated in situ carcinoma of the cervix, or completely treated and resolved non-metastatic squamous or basal cell carcinoma of the skin
(23) Known systemic hypersensitivity to dupilumab or the excipients of the drug product
(24) Patients having a contraindication to systemic corticosteroids (e.g., severe osteoporosis, adrenal insufficiency, Cushing's disease) that, in the investigator's judgment, would adversely affect the patient's participation in the study
(25) Severe concomitant illness(es) that, in the investigator's judgment, would adversely affect the patient's participation in the study. Examples include, but are not limited to, patients with short life expectancy, patients with uncontrolled diabetes (HbA1c ≥9%), patients with cardiovascular conditions (e.g., stage Ill or IV cardiac failure according to the New York Heart Association classification), severe renal conditions (e.g., patients on dialysis), debilitating neurological conditions (e.g., demyelinating diseases), active major autoimmune diseases (e.g., lupus, inflammatory bowel disease, rheumatoid arthritis, etc.), other severe endocrinological, gastrointestinal, hepatobiliary, metabolic, pulmonary, or lymphatic diseases.
(26) Any other medical or psychological condition (including relevant laboratory abnormalities at screening) that, in the opinion of the investigator, may suggest a new and/or insufficiently understood disease, may present an unreasonable risk to the study patient as a result of his/her participation in this clinical trial, may make patient's participation unreliable, or may interfere with study assessments
(27) History of alcohol or drug abuse within 2 years before the screening visit
(28) Patients who are committed to an institution by virtue of an order issued either by the judicial or the administrative authorities
(29) Pregnant or breastfeeding women or planning to become pregnant or breastfeed during the patient's participation in this study
(30) Women of childbearing potential (WOCBP) who are unwilling to practice highly effective contraception prior to the initial dose/start of the first treatment, during the study, and for at least 12 weeks after the last dose
(31) Patient is a member of the investigational team or his/her immediate family

### Study Treatment

The investigational and reference treatments for this study were as follows:
Dupilumab: loading dose of 600 mg administered SC, followed by 300 mg administered SC Q2W
Dupilumab-matching placebo (containing same formulation as dupilumab without active substance): loading dose administered SC, followed by SC q2w dosing

Background Treatment: All patients received a standard regimen of OCS (prednisone or prednisolone) on day 1/baseline visit to obtain control of disease. Specifically, patients with moderate BP (BPDAI activity score ≥24 and <60) received 0.5 mg/kg/day of prednisone (or prednisolone) and patients with severe BP (BPDAI activity score ≥60) received 0.75 mg/kg/day of prednisone (or prednisolone).

Per the article “Definitions and outcome measures for bullous pemphigoid: Recommendations by an international panel of experts” (Murrell et al., J Am Acad Dermatol 2012, 66:479-485), control of disease activity occurs when new lesions (e.g., blisters, urticarial plaques) cease to form and existing lesions begin to heal (e.g., show signs of epithelialization). Patients therefore were maintained on their assigned treatment level of OCS until they achieved control of disease activity. Control of disease activity was expected to occur by 2 weeks after randomization with the aforementioned doses of OCS, but some patients could be expected to take longer, usually no longer than 4 weeks.

For moderate BP patients not showing signs of disease control (e.g., continued blister or urticarial plaque formation) 2 weeks after randomization (week 2), the dose of OCS could be increased to 0.75 mg/kg/day of prednisone (or prednisolone).

It was recommended that tapering of OCS should begin when there has been 2 weeks of sustained control of disease activity. This coincides with the “end of the consolidation phase” which is defined as the time at which there has been control of disease activity for a minimum of 2 weeks and when tapering of OCS should begin (Murrell, supra). Oral corticosteroid tapering could therefore start at week 4 or up to week 6 depending on when initial control of disease activity was achieved.

Patients were to undergo a protocol-defined OCS tapering regimen as long as they maintained control of disease activity. The OCS taper employed in this study used a schedule typical for this disease, which is as follows:

For patients with moderate BP on prednisone (or prednisolone) 0.5 mg/kg/day:
0.3 mg/kg/day for 2 weeks,
0.2 mg/kg/day for 2 weeks,
0.1 mg/kg/day for 2 weeks,
0.05 mg/kg/day for 2 weeks, then discontinuation

For patients with severe BP on prednisone (or prednisolone) 0.75 mg/kg/day (or moderate BP who have been escalated to prednisone (or prednisolone) 0.75 mg/kg/day):
0.5 mg/kg/day for 2 weeks,
0.3 mg/kg/day for 2 weeks,
0.2 mg/kg/day for 2 weeks,
0.1 mg/kg/day for 2 weeks,
0.05 mg/kg/day for 2 weeks, then discontinuation

Rescue treatments: Patients requiring doses greater than 0.75 mg/kg/day of prednisone (or prednisolone) and those who did not achieve control of disease activity by week 4 were considered treatment failures. These patients could receive rescue treatment per investigator's discretion.

During the OCS taper period, patients who experienced a loss of control of disease activity (i.e., new lesions [e.g., blisters, urticarial plaques] begin to form and existing lesions do not heal) were considered treatment failures, but were allowed to increase the prednisone (or prednisolone) dose to the dose level where there was control of disease activity before resuming the predefined OCS tapering regimen. If a patient failed to complete the OCS tapering regimen (to a dose of 0.05 mg/kg/day or less) a second time, they were considered for rescue.

Following the OCS taper period, there was a remission period of at least 36 weeks where patients were observed for relapses off OCS. A relapse is defined as the appearance of 3 or more new lesions a month (blisters, eczematous lesions, or urticarial plaques) or at least 1 large (>10 cm diameter) eczematous lesion or urticarial plaque that does not heal within 1 week. Patients who experienced a relapse after completely tapering off OCS could be rescued, including with re-initiation of OCS. Rescue treatment could include treatment with high-potency topical corticosteroids, OCS (e.g., increases of OCS doses during the taper regimen, or re-initiation of OCS after tapering completely off OCS), and systemic non-steroidal immunosuppressive drugs or immunomodulating biologics. If topical or oral corticosteroids were used as rescue treatments, patients could continue study treatment. If a patient received rescue treatment with a systemic non-steroidal immunosuppressive drug or immunomodulating biologic, study treatment was permanently discontinued immediately.

### Study Variables

For this study, baseline characteristics include standard demography (e.g., age, race, weight, height, etc.), disease characteristics including medical history, and medication history for each patient. The efficacy variables include measurements or scores for individual patients for the following: BP clinical assessment (includes disease relapses), total cumulative dose of OCS, peak pruritus NRS, skin pain NRS, sleep quality NRS, BPDAI activity score, BPDAI pruritus, EQ-5D-3L, BSA, blister and urticaria/eczematous plaque count, ABQOL, PGADS, PGAT, and BP180 and BP230 autoantibody titers.

Body Surface Area (BSA): Body surface area affected by BP is assessed for each major section of the body (the possible highest score for each region is: head and neck [9%], anterior trunk [18%], back [18%], upper limbs [18%], lower limbs [36%], and genitals [1%]) and reported as a percentage of all major body sections combined. Patients will undergo this assessment at specified time points.

Pruritus Numerical Rating Scale: The Pruritus NRS is a simple assessment tool that patients use to report the intensity of their pruritus (itch) during a 24-hour recall period. Patients will complete the rating scale daily.

Skin Pain Numerical Rating Scale: Skin pain is measured using a validated skin pain NRS. This is an 11-point scale (0 to 10) in which 0 indicates no pain while 10 indicates worst pain possible. Patients will complete the scale at specified time points.

Sleep Numerical Rating Scale: Sleep quality is measured using a validated sleep quality NRS. This is an 11-point scale (0 to 10) in which 0 indicates worst possible sleep while 10 indicates best possible sleep. Patients will complete the scale at specified time points.

Bullous Pemphiqoid Disease Area Index Activity Score: The BPDAI activity score is a well-developed and validated instrument in BP patients (Wijayanti et al., Acta Derm Venereol 2017, 97:24-31). The total BPDAI activity score is the arithmetic sum of 3 subcomponents: cutaneous blisters/erosions, cutaneous urticaria/erythema, and mucosal blisters/erosions. Scores can range from 0 to 360 for BPDAI total activity (maximum 240 for total skin activity and 120 for mucosal activity), with higher scores indicating greater disease activity. The minimal clinically important difference (MCID) in the BPDAI activity score is 4 points for assessing clinical improvement and 3 points for deterioration. Patients will undergo this assessment at specified time points.

Bullous Pemphiqoid Disease Area Index Pruritus: A separate subjective component of the BPDAI, BPDAI Pruritus, is used to assess pruritus. The intensity of pruritus is subjectively graded using a visual analog scale where 0 represents no itch and 10 represents maximal itching. Patients mark an “x” to indicate severity of itching in the past 24 hours, in the past week, and in the past month, producing a total score out of 30. In cases where the patient was unable to reliably complete the grading (due to impaired mental functioning), pruritus was inferred from the degree of excoriations, also scored out of 30. However, for this study, patients should be the only ones completing this assessment. Patients will undergo this assessment at specified time points.

Patient Global Assessment of Disease Severity (PGADS): Patients rate their overall wellbeing based on a 5-point scale. Patients are asked: “Overall, how would you rate your bullous pemphigoid symptoms right now?” Response choices are: “No symptoms”, “Mild Symptoms”, “Moderate Symptoms”, “Severe Symptoms”, “Very Severe Symptoms”. Patients will undergo this assessment at specified time points.

Patient Global Assessment of Treatment (PGAT): Patients rate their satisfaction with the study treatment based on a 5-point scale. Patients are asked: “Compared to before you started the study, how would you rate your bullous pemphigoid symptoms now?” Response choices are: “Much Better”, “A Little Better”, “No Difference”, “A Little Worse”, “Much Worse”. Patients will undergo this assessment at specified time points.

Patient-Assessed EQ-5D-3L: The EQ-5D-3L is a standardized measure of health status developed by the EuroQOL Group to provide a simple, generic measure of health for clinical and economic appraisal. The EQ-5D-3L consists of 2 parts: the descriptive system and the EQ visual analogue scale (EQVAS). The EQ-5D-3L descriptive system comprises the following 5 dimensions: mobility, self-care, usual activities, pain/discomfort, and anxiety/depression. Each dimension has 3 levels of perceived problems: “no problem” (level 1), “some problems” (level 2), “extreme problems” (level 3). The respondent is asked to indicate his/her health state by ticking (or placing a cross) in the box against the most appropriate statement (i.e., no problems, some problems, or severe problems) in each of the 5 dimensions; this results in a 1-digit number expressing the level for that dimension. The digits for 5 dimensions can be combined in a 5-digit number describing the respondent's health state. The EQVAS records the respondent's self-rated health on a vertical, visual analogue scale where the endpoints are labeled “best imaginable health state (100)” and “worst imaginable health state (0).” This information can be used as a quantitative measure of health outcome as judged by the individual respondents. The questionnaire will be administered only to the subset of patients who fluently speak a language in which the questionnaire is presented (based on availability of validated translations in participating countries), at specified time points.

Autoimmune Bullous Disease Quality of Life: ABQOL has been shown to have good validity and reliability (Sebaratnam, et al., JAMA Dermatol 2013, 149:1186-1191). This questionnaire consists of 17 items, which encompass physical burden of the disease, psychiatric effects, and effects on daily life functioning. Each question ranges from 0 to 3 points, with higher scores indicating poorer quality of life. The maximum ABQOL score is 51. Patients will undergo this assessment at specified time points.

Pharmacodynamic and biomarker variables include biopsy and laboratory test results for individual patients for the following:

Serum Total IqE: Serum total IgE is a marker of type 2 inflammation. Dupilumab significantly suppressed total serum IgE in studies of adult patients with moderate-to-severe AD and is a pharmacodynamic marker of dupilumab activity.

Serum TARC (CCL17): Thymus and activation-regulated chemokine (TARC/CCL17) is a chemokine downstream of type 2 inflammation. Serum levels of TARC have also been reported to be elevated in BP patients, compared with healthy controls.

Serum or plasma PARC (CCL18): Pulmonary and activation-regulated chemokine (PARC/CCL18) is a chemotactic factor for Th2 cells and is also a downstream chemokine of the type 2 immune response. Serum PARC is highly elevated in patients with asthma and in allergic individuals. In one study, the levels of CCL18 in the serum of BP patients were found to be 84% higher than those normally observed in healthy individuals (Gunther, et al., Br J Dermatol 2009, 160:747-755).

Serum anti-BP180 and anti-BP230 autoantibodies of the IqG subclass concentration: A serologic characteristic of BP is the presence of circulating autoantibodies in patient serum, which are mostly against BP180 (collagen XVII) and BP230 (hemidesmosomal proteins). NC16A, a non-collagenous stretch of the BP180 ectodomain, is the primary target of the pathogenic IgG antibodies. Autoantibodies from BP patients react with BP180, which leads to the degradation of the basement membrane, and this has been regarded as a central event in BP pathogenesis. IgG autoantibodies against BP180 are the ones first to be detected, followed by IgG autoantibodies against BP230. Concentrations of anti-BP180 and anti-BP230 are elevated during active disease and decrease with resolution of BP.

Serum anti-BP180 and anti-BP230 autoantibodies of the IqE subclass titers: IgE autoantibodies to BP180 and BP230 are detected at high frequencies in BP. One study suggests that IgE anti-BP230 levels have a strong association with local eosinophil accumulation and are postulated to play a role in attracting eosinophils to the skin lesions (Ishiura, et al., J Dermatol Sci 2008, 49:153-161). Another study links the presence or levels of IgE anti-BP180 Abs with broader skin lesions. The study also suggests that patients with BP positive for IgE anti-BP180 antibodies require a longer duration for remission, a higher dosage of prednisolone, and more intensive therapies for remission (Iwata, et al., Arch Dermatol 2008, 144:41-48).

Serum anti-BP180 and anti-BP230 autoantibodies of the IgG4 subclass titers: In addition to IgE and IgG, autoantibodies of the IgG4 subclasses have been found to be upregulated in BP and have been associated with onset of BP flares (Dopp, et al., J Am Acad Dermatol 2000, 42:577-583).

### Results

The study enrolled 106 subjects who were randomized to receive dupilumab 300 mg (n=53) every two weeks after an initial loading dose or placebo (n=53), both added to standard-of-care oral corticosteroids (OCS). During treatment, all subjects underwent a protocol-defined OCS tapering regimen if control of disease activity was maintained. Baseline demographics (Table 2) were generally balanced between treatment arms except for more female subjects in the dupilumab arm as compared to placebo. Baseline disease characteristics are shown in Table 3 below. The study population consisted of elderly participants with moderate to severe disease and high symptom burden. Nearly two-thirds of the patients in the trial had new onset of the disease.

TABLE 2
Baseline Demographics

| | Placebo | Dupilumab | Total |
|---|---|---|---|
| | (N = 53) | (N = 53) | (N = 106) |
| Age (years), mean (SD) | 71.5 (9.52) | 71.2 (10.01) | 71.3 (9.72) |
| Age group, n (%) | | | |
| <65 years | 11 (20.8%) | 13 (24.5%) | 24 (22.6%) |
| ≥65 years to <75 years | 20 (37.7%) | 18 (34.0%) | 38 (35.8%) |
| ≥75 years | 22 (41.5%) | 22 (41.5%) | 44 (41.5%) |
| Sex, n (%) | | | |
| Male | 29 (54.7%) | 21 (39.6%) | 50 (47.2%) |
| Female | 24 (45.3%) | 32 (60.4%) | 56 (52.8%) |
| Ethnicity, n (%) | | | |
| Hispanic or Latino | 3 (5.7%) | 0 | 3 (2.8%) |
| Not Hispanic or Latino | 42 (79.2%) | 43 (81.1%) | 85 (80.2%) |
| Unknown | 1 (1.9%) | 2 (3.8%) | 3 (2.8%) |
| Not Reported | 7 (13.2%) | 8 (15.1%) | 15 (14.2%) |
| Race, n (%) | | | |
| White | 37 (69.8%) | 35 (66.0%) | 72 (67.9%) |
| Black or African American | 1 (1.9%) | 1 (1.9%) | 2 (1.9%) |
| Asian | 7 (13.2%) | 9 (17.0%) | 16 (15.1%) |
| American Indian or Alaska Native | 0 | 0 | 0 |
| Native Hawaiian or Other Pacific Islander | 0 | 0 | 0 |
| Other | 2 (3.8%) | 0 | 2 (1.9%) |
| Not Reported | 6 (11.3%) | 8 (15.1%) | 14 (13.2%) |
| Region, n (%) | | | |
| North America | 12 (22.6) | 12 (22.6) | |
| Europe | 34 (64.2) | 32 (60.4) | |
| Asia | 7 (13.2) | 9 (17.0) | |
| Weight (kg), mean (SD) | 78.4 (15.77) | 76.6 (20.44) | 77.5 (18.19) |

TABLE 3
Baseline Disease Characteristics

| | Placebo | Dupilumab | Total |
|---|---|---|---|
| | (N = 53) | (N = 53) | (N = 106) |
| Duration of BP (years) | 0.9 (1.77) | 1.0 (1.41) | 1.0 (1.59) |
| Mean (SD)/Median (IQR) | 0.3 (0.1:0.7) | 0.5 (0.2:1.1) | 0.3 (0.2:0.8) |
| BP disease status at baseline, n (%) | | | |
| New onset | 34 (64.2%) | 35 (66.0%) | 69 (65.1%) |
| Relapsed | 19 (35.8%) | 18 (34.0%) | 37 (34.9%) |
| Age at BP onset (years), mean (SD) | 71.1 (9.52) | 70.9 (10.13) | 71.0 (9.79) |
| Used prior steroids/immunosuppressants, n (%) | 32 (60.4%) | 30 (56.6%) | 62 (58.5%) |
| Baseline disease severity [BPDAI activity | | | |
| score range 0-360] | | | |
| Moderate (BPDAI activity score ≥24 to <60), n (%) | 36 (67.9%) | 34 (64.2%) | 70 (66.0%) |
| Severe (BPDAI activity score ≥60), n (%) | 17 (32.1%) | 19 (35.8%) | 36 (34.0%) |
| Baseline BPDAI activity score [range 0-360] | 58.5 (28.47) | 62.5 (31.58) | 60.5 (29.99) |
| Mean (SD)/Median (IQR) | 57.0 (35.0:72.0) | 56.0 (42.0:74.0) | 57.0 (39.0:72.0) |
| Baseline peak pruritus NRS [range 0-10] | 7.4 (1.54) | 7.6 (1.64) | 7.5 (1.59) |
| Mean (SD)/Median (IQR) | 7.8 (6.3:8.5) | 7.7 (6.6:9.0) | 7.7 (6.4:8.8) |
| Baseline % BSA affected by BP, mean (SD) | 32.2 (21.63) | 29.4 (19.13) | 30.8 (20.37) |
| Baseline ABQOL score [range 0-51], mean (SD) | 21.7 (9.12) | 22.9 (9.82) | 22.3 (9.45) |
| Baseline skin pain NRS score, mean (SD) | 5.8 (2.52) | 6.4 (2.22) | 6.1 (2.38) |
| Baseline serum anti-BP180 IgG (U/mL) | 320.6 (1004.3) | 153.5 (310.8) | 236.0 (739.6) |
| Mean (SD)/Median (IQR) | 61.5 (17.0:99.5) | 72.0 (35.0:99.0) | 64.0 (23.0:99.0) |
| Baseline serum anti-BP230 IgG (U/mL) | 40.1 (36.3) | 82.4 (207.3) | 61.5 (150.3) |
| Mean (SD)/Median (IQR) | 33.0 (4.5:72.0) | 6.0 (2.0:65.0) | 14.0 (3.0:69.0) |
| ABQOL score, mean (SDS) | 21.7 (9.1) | 22.9 (9.8) | |
| Serum total IgE, mean (SD), U/mL | 1861 (3102.7) | 1502 (3041.5) | |
| Blood eosinophils, mean (SD), 10⁹/L | 0.943 (1.2) | 1.15 (1.5) | |

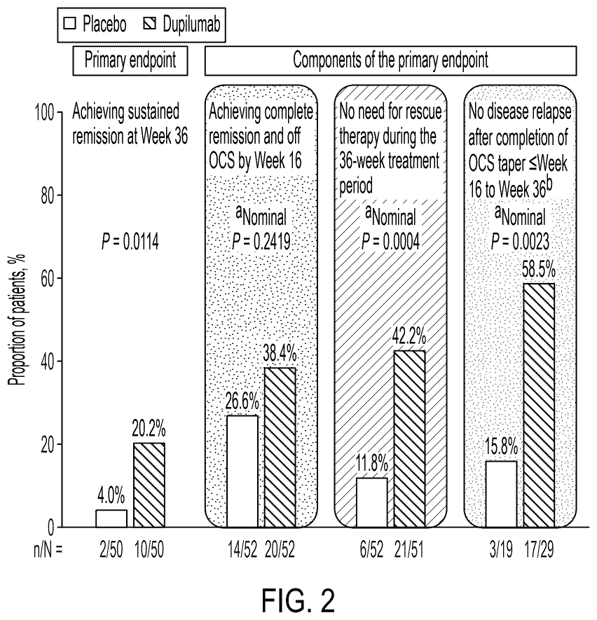

In the trial, dupilumab met the primary and all key secondary endpoints. Five times more dupilumab-treated participants achieved the primary endpoint of sustained disease remission at week 36 compared to those on placebo (FIG. 2). Sustained disease remission was defined as the achievement of complete remission and off OCS no later than week 16, absence of disease relapse from the time the subject completed OCS taper (no later than week 16) to week 36, and absence of need for rescue therapy during the 36-week treatment period. In the dupilumab arm, 20.2% of subjects achieved sustained remission at week 36, as compared to 4.0% of subjects in the placebo arm (p=0.0114). Components of the primary endpoint show a substantial benefit of dupilumab in preventing disease relapse post-OCS taper and reduced need for rescue therapy (Table 4 and FIG. 2). In the dupilumab arm, 42% of subjects did not use any rescue medications during the 36-week treatment period, vs. 12% of placebo-treated subjects.

TABLE 4
Components of Primary Endpoint (Sustained Remission at Week 36)

| | | | Treatment | |
|---|---|---|---|---|
| | Placebo | Dupilumab | Difference | Nominal |
| Components of Primary Endpoint | n = 53 | n = 53 | (95% CI) | p-value |
| Achievement of complete | 26.6% | 38.4% | 11.8 | 0.2419 |
| remission and off OCS no | [Obs n = 14/52] | [Obs n = 20/52] | (−6.16, 29.07) | |
| later than week 16 | | | | |
| Absence of disease relapse from | 15.8% | 58.5% | 42.7 | 0.0023 |
| the time subject completed OCS | [Obs n = 3/19] | [Obs n = 17/29] | (15.60, 62.93) | |
| taper (no later than week 16) to | | | | |
| week 36* | | | | |
| Absence of need for rescue | 11.8% | 42.2% | 30.4 | 0.0004 |
| therapy during the 36-week | [Obs n = 6/52] | [Obs n = 21/51] | (13.92, 45.77) | |
| treatment period | | | | |

*Analysis conducted among patients who completed the OCS taper

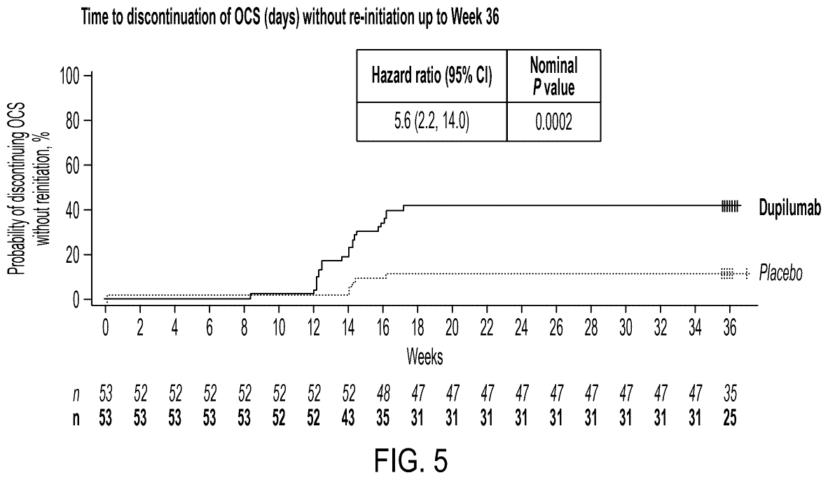

Treatment with dupilumab significantly delayed the time to first rescue medication use (171.5 days for dupilumab-treated subjects vs. 123.8 days for placebo-treated subjects [p=0.0016]). Additionally, the mean total cumulative dose of OCS was reduced by 1678 mg (5646 mg for dupilumab-treated subjects, compared to 7324 mg for placebo-treated subjects) [p=0.0220]). Dupilumab-treated subjects also had a significantly longer duration of complete remission off OCS (39.9 days for the dupilumab arm vs. 12.5 days for the placebo arm; p=0.0072) and a 54% lower risk of rescue use (P=0.0016). Dupilumab-treated subjects were more likely to discontinue OCS and remain off OCS (FIG. 5).

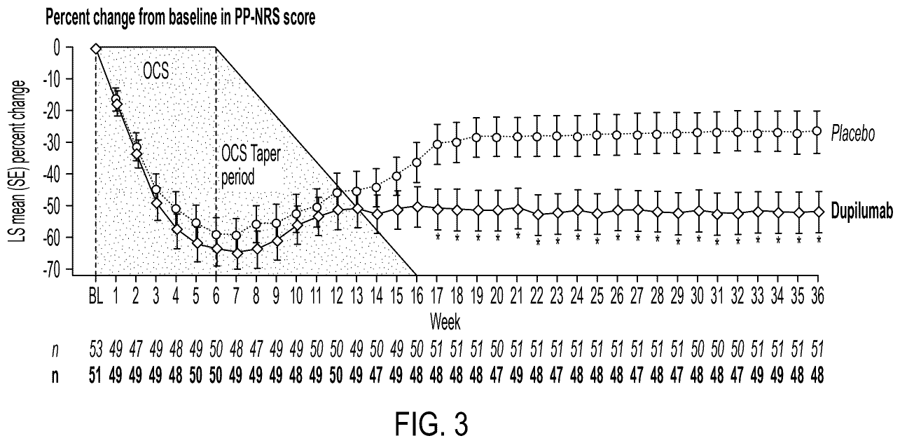

A significant proportion of dupilumab-treated subjects experienced a clinically meaningful itch reduction, as measured by an improvement (reduction) of weekly average of daily peak pruritus NRS 4 from baseline to week 36 (39.8% for dupilumab-treated subjects, compared to 10.6% for placebo-treated subjects; p=0.0006). See also, FIG. 3. Dupilumab-treated subjects also had a significantly greater percent reduction in weekly average of daily peak pruritus NRS from baseline to week 36 (−26.6% for dupilumab-treated subjects, as compared to −25.7% for placebo-treated subjects; p=0.0021).

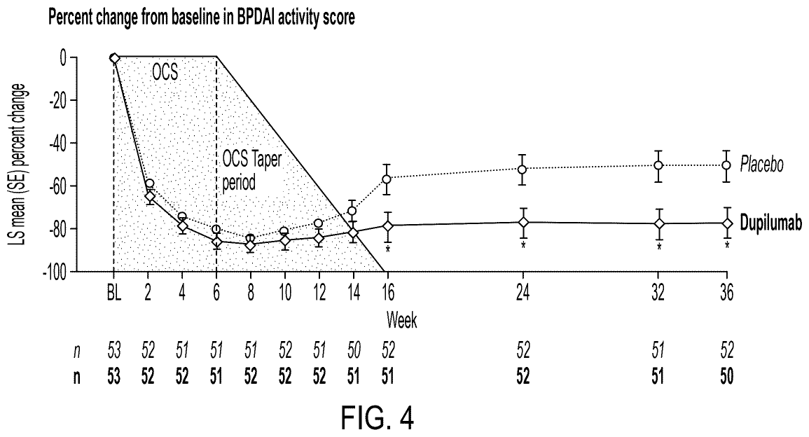

Additionally, dupilumab-treated subjects exhibited a significantly greater reduction in BP disease activity. Percent change in BPDAI activity score from baseline to week 36 was −76.7% in the dupilumab arm as compared to −50.5% in the placebo arm (p=0.0021). See also, FIG. 4. In the dupilumab arm, 40.5% of subjects achieved a reduction in BPDAI activity score of at least 90% from baseline to week 36, as compared to 9.8% of subjects in the placebo arm (p=0.0003).

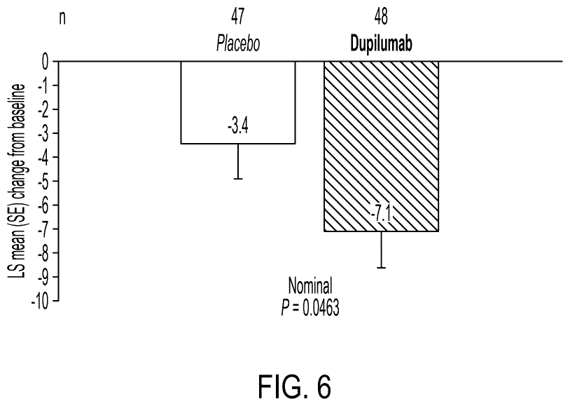

Dupilumab-treated subjects also achieved greater improvements in health-related quality of life, as measured by the ABQOL questionnaire. At Week 36, dupilumab-treated subjects had a mean change in ABQOL score of −7.1, as compared to only −3.4 for placebo-treated subjects (p=0.0463) (FIG. 6).

### Safety

Safety results were generally consistent with the known safety profile of dupilumab in its approved dermatological indications. More placebo (30.2%) than dupilumab-treated (22.6%) patients reported SAEs. Overall rates of adverse events (AE) were 96% for dupilumab and 96% for placebo. AEs more commonly observed with dupilumab compared to placebo in ≥5% of patients included peripheral edema (15% dupilumab, n=8; 9% placebo, n=5), arthralgia (9% dupilumab, n=5; 6% placebo, n=3), back pain (8% dupilumab, n=4; 4% placebo, n=2), blurred vision (8% dupilumab, n=4; 0% placebo), hypertension (8% dupilumab, n=4; 6% placebo, n=3), asthma (8% dupilumab, n=4; 2% placebo, n=1), conjunctivitis (8% dupilumab, n=4; 0% placebo), constipation (8% dupilumab, n=4; 2% placebo, n=1), upper respiratory tract infection (6% dupilumab, n=3; 2% placebo, n=1), limb injury (6% dupilumab, n=3; 4% placebo, n=2), and insomnia (6% dupilumab, n=3; 4% placebo, n=2). There were no AEs leading to death in the dupilumab group and 2 AEs leading to death in the placebo group (4%).

### CONCLUSION

Dupilumab demonstrated significant benefits across multiple disease aspects in BP including achieving sustained remission, reducing disease activity and itch, and providing corticosteroid-sparing effects. Overall safety was consistent with the known dupilumab safety profile.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for treating bullous pemphigoid (BP) or reducing or ameliorating one or more symptoms of BP in a subject in need thereof, the method comprising:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject has (i) a baseline Bullous Pemphigoid Disease Area Index (BPDAI) activity score ≥24, and (ii) a baseline pruritus Numerical Rating Score (NRS) 4.

2. The method of claim 1, wherein the subject is on a background therapy comprising an oral corticosteroid.

3. A method for reducing or eliminating the need for oral corticosteroid therapy in a subject having bullous pemphigoid, the method comprising:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject has (i) a baseline Bullous Pemphigoid Disease Area Index (BPDAI) activity score ≥24, and (ii) a baseline pruritus Numerical Rating Score (NRS) 4; and wherein the subject is on a background therapy comprising an oral corticosteroid.

4. A method for achieving sustained disease remission in the absence of oral corticosteroid therapy in a subject having bullous pemphigoid, the method comprising:
administering to the subject an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8;
wherein the subject has (i) a baseline Bullous Pemphigoid Disease Area Index (BPDAI) activity score ≥24, and (ii) a baseline pruritus Numerical Rating Score (NRS) 4.

5. The method of claim 4, wherein the subject is on a background therapy comprising an oral corticosteroid at the start of treatment with the IL-4R antagonist, and wherein the method further comprises discontinuing the oral corticosteroid therapy.

6. The method of any one of claims 1 to 5, wherein the subject has a baseline BPDAI activity score ≥24 and <60 and is on a background therapy of prednisone or prednisolone 0.5 mg/kg/day.

7. The method of any one of claims 1 to 5, wherein the subject has a baseline BPDAI activity score ≥60 and is on a background therapy of prednisone or prednisolone 0.75 mg/kg/day.

8. The method of any one of claims 1 to 7, wherein the subject was previously treated with an immunosuppressant.

9. The method of any one of claims 1 to 8, wherein the subject has had prior systemic corticosteroid use for BP.

10. The method of any one of claims 1 to 9, wherein the subject has a baseline Peak Pruritus NRS ≥7.

11. The method of any one of claims 1 to 10, wherein the subject is an adult.

12. The method of any one of claims 1 to 11, wherein at least 8 doses of the IL-4R antagonist are administered to the subject.

13. The method of any one of claims 1 to 12, wherein treatment with the IL-4R antagonist results in:
achievement of sustained remission by week 36 from the start of treatment;
reduction in total cumulative dose of OCS administered from baseline to week 36 from the start of treatment;
reduction in weekly average of daily peak pruritus numerical rating score (NRS) from baseline to week 36 from the start of treatment;
reduction in BPDAI score from baseline to week 36 from the start of treatment;
increase in time to first use of rescue medication from baseline to week 36 from the start of treatment;
reduction in BP180 autoantibody titer and/or BP230 autoantibody titer from baseline to week 36 from the start of treatment;
improvement in autoimmune bullous disease quality of life (ABQOL) from baseline to week 36 from the start of treatment; and/or
reduction in percent body surface area (BSA) of BP involvement from baseline to week 36 from the start of treatment.

14. The method of any one of claims 1 to 13, wherein treatment with the IL-4R antagonist results in a reduction of 4 points in peak pruritus NRS from baseline to week 36 from the start of treatment.

15. The method of any one of claims 1 to 14, wherein treatment with the IL-4R antagonist results in:
a reduction in BPDAI activity score ≥50% from baseline to week 36;
a reduction in BPDAI activity score ≥75% from baseline to week 36;
a reduction in BPDAI activity score ≥90% from baseline to week 36;
a reduction in BPDAI activity score ≥50% from baseline to week 52;
a reduction in BPDAI activity score ≥75% from baseline to week 52; and/or
a reduction in BPDAI activity score ≥90% from baseline to week 52.

16. The method of any one of claims 1 to 15, wherein the IL-4R antagonist is administered to the subject as an initial dose followed by one or more secondary doses, wherein the initial dose is about 600 mg and each secondary dose is about 300 mg.

17. The method of claim 16, wherein the secondary dose is administered two weeks after the immediately preceding dose (Q2W).

18. The method of any one of claims 1 to 17, wherein the IL-4R antagonist is administered subcutaneously.

19. The method of any one of claims 1 to 18, wherein the anti-IL-4R antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:2.

20. The method of any one of claims 1 to 19, wherein the anti-IL-4R antibody is a full antibody.

21. The method of claim 20, wherein the full antibody is an IgG antibody.

22. The method of claim 20 or 21, wherein the full antibody is an IgG4 antibody.

23. The method of any one of claims 1 to 22, wherein the anti-IL-4R antibody comprises a heavy chain comprising the amino acid sequence of SEQ ID NO:9 and a light chain comprising the amino acid sequence of SEQ ID NO:10.

24. The method of any one of claims 1 to 23, wherein the IL-4R antagonist is dupilumab.

25. The method of any one of claims 1 to 24, wherein the IL-4R antagonist is contained in a container selected from the group consisting of a glass vial, a syringe, a pre-filled syringe, a pen delivery device, and an autoinjector.

26. The method of claim 25, wherein the IL-4R antagonist is contained in a pre-filled syringe.

27. The method of claim 26, wherein the pre-filled syringe is a single-dose pre-filled syringe.

28. The method of claim 25, wherein the IL-4R antagonist is contained in an autoinjector.

29. The method of claim 25, wherein the IL-4R antagonist is contained in a pen delivery device.

30. The method of any one of claims 1-29, wherein the IL-4R antagonist is administered to the subject in combination with a tapering course of oral corticosteroids.


SEQUENCE LISTING (366 sequences)

SEQ ID NO: 1 - protein, 124 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, unidentified [sequence omitted]

SEQ ID NO: 3 - protein, 8 aa, unidentified
GFTFRDYA

SEQ ID NO: 4 - protein, 8 aa, unidentified
ISGSGGNT

SEQ ID NO: 5 - protein, 18 aa, unidentified
AKDRLSITIRPRYYGLDV

SEQ ID NO: 6 - protein, 11 aa, unidentified
QSLLYSIGYNY

SEQ ID NO: 7 - skipped

SEQ ID NO: 8 - protein, 9 aa, unidentified
MQALQTPYT

SEQ ID NO: 9 - protein, 451 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, unidentified
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, unidentified
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, unidentified
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, unidentified
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, unidentified
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, unidentified
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, unidentified
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, unidentified
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, unidentified
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, unidentified
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, unidentified
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, unidentified
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, unidentified
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, unidentified
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, unidentified
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, unidentified
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, unidentified
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, unidentified
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, unidentified
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, unidentified
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, unidentified
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, unidentified
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, unidentified
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, unidentified
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, unidentified
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, unidentified
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, unidentified
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, unidentified
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, unidentified
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, unidentified
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, unidentified
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, unidentified
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, unidentified; identical to SEQ ID NO: 69

SEQ ID NO: 74 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, unidentified; identical to SEQ ID NO: 74

SEQ ID NO: 77 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, unidentified; identical to SEQ ID NO: 74

SEQ ID NO: 81 - protein, 116 aa, unidentified; identical to SEQ ID NO: 79

SEQ ID NO: 82 - protein, 111 aa, unidentified; identical to SEQ ID NO: 78

SEQ ID NO: 83 - protein, 116 aa, unidentified; identical to SEQ ID NO: 69

SEQ ID NO: 84 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, unidentified; identical to SEQ ID NO: 75

SEQ ID NO: 86 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, unidentified; identical to SEQ ID NO: 87

SEQ ID NO: 90 - protein, 111 aa, unidentified; identical to SEQ ID NO: 84

SEQ ID NO: 91 - protein, 116 aa, unidentified; identical to SEQ ID NO: 75

SEQ ID NO: 92 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, unidentified; identical to SEQ ID NO: 69

SEQ ID NO: 94 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, unidentified; identical to SEQ ID NO: 69

SEQ ID NO: 96 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, unidentified; identical to SEQ ID NO: 69

SEQ ID NO: 98 - protein, 111 aa, unidentified; identical to SEQ ID NO: 78

SEQ ID NO: 99 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, unidentified; identical to SEQ ID NO: 99

SEQ ID NO: 102 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, unidentified; identical to SEQ ID NO: 99

SEQ ID NO: 104 - protein, 111 aa, unidentified; identical to SEQ ID NO: 78

SEQ ID NO: 105 - protein, 116 aa, unidentified; identical to SEQ ID NO: 77

SEQ ID NO: 106 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, unidentified
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, unidentified; identical to SEQ ID NO: 77

SEQ ID NO: 110 - protein, 111 aa, unidentified
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, unidentified
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, unidentified
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, unidentified
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, unidentified
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, unidentified; identical to SEQ ID NO: 77

SEQ ID NO: 118 - protein, 111 aa, unidentified; identical to SEQ ID NO: 114

SEQ ID NO: 119 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, unidentified
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, unidentified
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, unidentified; identical to SEQ ID NO: 77

SEQ ID NO: 126 - protein, 111 aa, unidentified
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, unidentified; identical to SEQ ID NO: 77

SEQ ID NO: 128 - protein, 111 aa, unidentified
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, unidentified; identical to SEQ ID NO: 77

SEQ ID NO: 130 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, unidentified
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, unidentified
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, unidentified
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, unidentified; identical to SEQ ID NO: 114

SEQ ID NO: 135 - protein, 116 aa, unidentified; identical to SEQ ID NO: 77

SEQ ID NO: 136 - protein, 111 aa, unidentified; identical to SEQ ID NO: 124

SEQ ID NO: 137 - protein, 116 aa, unidentified; identical to SEQ ID NO: 107

SEQ ID NO: 138 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, unidentified
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, unidentified
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, unidentified; identical to SEQ ID NO: 107

SEQ ID NO: 142 - protein, 111 aa, unidentified; identical to SEQ ID NO: 114

SEQ ID NO: 143 - protein, 116 aa, unidentified; identical to SEQ ID NO: 113

SEQ ID NO: 144 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, unidentified; identical to SEQ ID NO: 77

SEQ ID NO: 146 - protein, 111 aa, unidentified; identical to SEQ ID NO: 138

SEQ ID NO: 147 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, unidentified
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, unidentified
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, unidentified
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, unidentified
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, unidentified
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, unidentified
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, unidentified
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, unidentified
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, unidentified
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, unidentified
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, unidentified
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, unidentified
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, unidentified
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, unidentified
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, unidentified
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, unidentified
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, unidentified
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, unidentified
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, unidentified
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, unidentified
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, unidentified
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, unidentified
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, unidentified
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, unidentified
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, unidentified
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, unidentified
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, unidentified
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, unidentified
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, unidentified
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, unidentified; identical to SEQ ID NO: 177

SEQ ID NO: 179 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, unidentified; identical to SEQ ID NO: 177

SEQ ID NO: 182 - protein, 111 aa, unidentified
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, unidentified
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, unidentified
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, unidentified
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, unidentified
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, unidentified
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, unidentified
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, unidentified
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, unidentified
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, unidentified
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, unidentified
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, unidentified
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, unidentified
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, unidentified
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, unidentified
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, unidentified
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, unidentified
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, unidentified
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, unidentified
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, unidentified
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, unidentified
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, unidentified
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, unidentified
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, unidentified
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, unidentified
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, unidentified
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, unidentified
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, unidentified
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, unidentified
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, unidentified
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, unidentified
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, unidentified
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, unidentified
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, unidentified
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, unidentified
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, unidentified
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, unidentified; identical to SEQ ID NO: 210

SEQ ID NO: 219 - protein, 120 aa, unidentified
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, unidentified
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, unidentified
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, unidentified
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, unidentified
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, unidentified
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, unidentified
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, unidentified
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, unidentified
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, unidentified; identical to SEQ ID NO: 227

SEQ ID NO: 229 - protein, 108 aa, unidentified
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, unidentified; identical to SEQ ID NO: 229

SEQ ID NO: 231 - protein, 108 aa, unidentified; identical to SEQ ID NO: 227

SEQ ID NO: 232 - protein, 108 aa, unidentified; identical to SEQ ID NO: 229

SEQ ID NO: 233 - protein, 108 aa, unidentified
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, unidentified
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, unidentified
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, unidentified
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, unidentified
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, unidentified
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, unidentified
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, unidentified
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, unidentified; identical to SEQ ID NO: 234

SEQ ID NO: 242 - protein, 108 aa, unidentified
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, unidentified
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, unidentified
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, unidentified
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, unidentified
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, unidentified
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, unidentified
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, unidentified
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, unidentified
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, unidentified
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, unidentified
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, unidentified
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, unidentified
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, unidentified
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, unidentified
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, unidentified; identical to SEQ ID NO: 254

SEQ ID NO: 258 - protein, 118 aa, unidentified
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, unidentified
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, unidentified
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, unidentified
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, unidentified
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, unidentified
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, unidentified
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, unidentified; identical to SEQ ID NO: 261

SEQ ID NO: 266 - protein, 111 aa, unidentified
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, unidentified
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, unidentified; identical to SEQ ID NO: 260

SEQ ID NO: 269 - protein, 107 aa, unidentified
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, unidentified
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, unidentified
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, unidentified
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, unidentified
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, unidentified
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, unidentified
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

SEQ ID NO: 276 - protein, 118 aa, unidentified
QVQLQQSGTELVKPGASVKMSCKASVNTFTGYNMHWIKQTPGQGLEWIGGLHPGNGDSSYNQKFKGRATLTDKSSNTAYMQLSSLTSEDSAVYYCALTTAGRAWFPYWGQGTLVTVSA

SEQ ID NO: 277 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEIK

SEQ ID NO: 278 - protein, 119 aa, unidentified
QVQLQQSGTELVKPGASVKMSCKASVNTFTGYNMHWIKQTPGQGLEWIGGLHPGNGDSSYNQKFKGRATLTADKSSNTAYMQLSSLTSEDSAVYYCALTTAGRAWFAYWGQGTLVTVSA

SEQ ID NO: 279 - protein, 106 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLHTFGGGTKLEIK

SEQ ID NO: 280 - protein, 119 aa, unidentified
QVQLQQSGAELVKPGASVKMSCKASVNTFAGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 281 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 282 - protein, 119 aa, unidentified
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 283 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLELK

SEQ ID NO: 284 - protein, 119 aa, unidentified
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFPYWGQGTLVTVSA

SEQ ID NO: 285 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 286 - protein, 119 aa, unidentified
QVQLQQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 287 - protein, 107 aa, unidentified; identical to SEQ ID NO: 285

SEQ ID NO: 288 - protein, 119 aa, unidentified
EVQLQQSGAELVKPGASVKMSCKASVNIFTGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 289 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 290 - protein, 119 aa, unidentified
EVKLVESGGGLVKPGGSLKLSCAASGFTFSRYAMSWVRQTPEKRLEWVATISSGGSYTNYADSVKGRFTISRDNVKNTLYLQMNSLRAEDTAVYYCARATARATEFAYWGQGTLVTVSS

SEQ ID NO: 291 - protein, 106 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEL

SEQ ID NO: 292 - protein, 119 aa, unidentified; identical to SEQ ID NO: 288

SEQ ID NO: 293 - protein, 107 aa, unidentified
DIQMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 294 - protein, 119 aa, unidentified
EVQLKESGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 295 - protein, 107 aa, unidentified; identical to SEQ ID NO: 293

SEQ ID NO: 296 - protein, 119 aa, unidentified
QVQLKQSGAELVKPGASVKMSCTASVNIFTGYNMHWIKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 297 - protein, 107 aa, unidentified
DIQMNQSTSSLSASLGDRVTISCRASQDISNYLNWYQQKPDGTIKLLIYYTSRLYSGVPSRFSGSGSGTDYSLTISNLEEEDIATYFCQQGNTIPYTFGGGTKLEIK

SEQ ID NO: 298 - protein, 119 aa, unidentified
EVQLQQSGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 299 - protein, 107 aa, unidentified
DIQMTQSTSSLSASLGDRVTISCRASQDISNYLNWYQQKPDGTIKLLIYYTSRLHSGVPSRFSGSGSGTDYSLTISNLEEEDIATYFCQQGNTLPWTFGGGTKLEIK

SEQ ID NO: 300 - protein, 119 aa, unidentified
EVQLQQSGAELVKPGASVKMSCKASVNIFTSYNMHWVKQTPGQGLEWIGGLHPGNGDTSYNQKFKGKATLTADKSSSTAYMQVSMLTSEDSAVYYCVLTTAGRAWFAYWGQGTLVTVSA

SEQ ID NO: 301 - protein, 107 aa, unidentified; identical to SEQ ID NO: 299

SEQ ID NO: 302 - protein, 119 aa, unidentified
QVQLQESGAELVKPGASVKMSCKASVNTFTGYNMHWVKQTPGQGLEWIGGLHPGNGDTSYNQKFKGKATLTADRSSNTAYMQLSSLTSEDSAVYYCALTTAGRAWFAYWGQGTLVTVSA

SEQ ID NO: 303 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 304 - protein, 119 aa, unidentified
QVQLKQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 305 - protein, 107 aa, unidentified
DIQMTQSTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 306 - protein, 119 aa, unidentified
QVQLKQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 307 - protein, 107 aa, unidentified
DIQMNQSTSSLSASLGDRVTISCRASQDISNYLNWYQQKPDGTIKLLIYYTSRLYSGVPSRFSGSGSGTDYSLTISNLEEEDIATYFCQQGNTLPYTFGGGTKLEIK

SEQ ID NO: 308 - protein, 119 aa, unidentified
QVQLQQSGAELVKPGASVKMSCKASVNTITGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 309 - protein, 108 aa, unidentified
DIKMTQSTSSLSASLGDRVTTSCRASQDISNYLNWYQQKPDGTVKLLIYYTSRLHSGVPSRFSGSGSGTDYSLTISNLEEEDIATYFCQQGNTLPPWTFGGGTKLEIK

SEQ ID NO: 310 - protein, 119 aa, unidentified
EVQLVESGGGLVKPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 311 - protein, 119 aa, unidentified
EVQLLESGGGLVQPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 312 - protein, 119 aa, unidentified
QVQLVQSGAELKKPGASVKVSCKASGNTFTGYNMHWIQQSPGQGLEWMGGLHPGNGDSSYNQKFQGRVTLTADKSSNTAYMELSSLRSEDSAVYYCALTTAGRAWFPYWGQGTLVTVSS

SEQ ID NO: 313 - protein, 119 aa, unidentified
QVQLVQSGAEVKKPGASVKMSCKASVYTFTGYNMHWIQQSPGQGLEWMGGLHPGNGDSSYNQKFQGRATLTADKSSNTAYMELSSLRSEDSAVYYCALTTAGRAWFPYWGQGTLVTVSS

SEQ ID NO: 314 - protein, 119 aa, unidentified
QVQLVQSGAEVKKPGASVKVSCKASGNIFTGYNMHWVRQAPGQGLEWMGGLHPGNGDSSYNQKFQGRVTLTADKSSNTAYMELSSLRSEDSAVYYCALTTAGRAWFAYWGQGTLVTVSS

SEQ ID NO: 315 - protein, 119 aa, unidentified
QVQLVQSGAEVKKPGASVKMSCKASVYTFTGYNMHWVRQAPGQGLEWIGGLHPGNGDSSYNQKFQGRATLTADKSSNTAYMELSSLRSEDSAVYYCALTTAGRAWFAYWGQGTLVTVSS

SEQ ID NO: 316 - protein, 119 aa, unidentified
EVQLVQSGAEVKKPGATVKISCKASGNIFTSYNMHWVRQAPGQGLEWMGGLHPGNGDTSYNQKFQGRVTLTADKSSSTAYMELSSLRSEDTAVYYCVLTTAGRAWFAYWGQGTLVTVSS

SEQ ID NO: 317 - protein, 119 aa, unidentified
EVQLVQSGAEVKKPGATVKMSCKASVYTFTSYNMHWVRQAPGQGLEWIGGLHPGNGDTSYNQKFKGRATLTADKSSSTAYMELSSLRSEDTAVYYCVLTTAGRAWFAYWGQGTLVTVSS

SEQ ID NO: 318 - protein, 107 aa, unidentified
DIQMTQSPSSLSASVGDRVTITCQASQDISNYLNWYQQKPGKAPKLLIYYTSRLHSGVPSRFSGSGSGTDYTFTISSLQPEDIATYFCQQGNTLPWTFGGGTKVEIK

SEQ ID NO: 319 - protein, 107 aa, unidentified
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPWTFGGGTKVEIK

SEQ ID NO: 320 - protein, 107 aa, unidentified
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPLTFGGGTKVEIK

SEQ ID NO: 321 - protein, 107 aa, unidentified
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPWTFGGGTKVEIK

SEQ ID NO: 322 - protein, 115 aa, unidentified
EVQLQQSGPELVKPGASVKISCKTSGYTFTEYTIHWVKQNHGKSLEWIGGINPNNGGTVYNQNFKGKATLVDKSSSTAYMELRSLTSEDSAVYYCARVRRGMDYWGQGTSVTVSS

SEQ ID NO: 323 - protein, 107 aa, unidentified
DIVMTQSHKFMSTSVGDRVSITCKASQDVTTAVAWYQQKPGQSPKLLIYSASYRYTGVPDRFTGSGSGTDFTFTISSVQAEDLAVYYCQQHYSAPWTFGGGTNLEIK

SEQ ID NO: 324 - protein, 116 aa, unidentified
QVQLQQSGAEVVKPGASVKISCKTSGYTFTEYTIHWVKQAHGQSLEWIGGINPNNGGTVYNQKFQGKATLTVDKSTSTAYMELRSLTSEDTAVYYCARVRRGMDYWGQGTSVTVSS

SEQ ID NO: 325 - protein, 107 aa, unidentified
DIQMTQSPKSTSTSVGDRVTITCRASQDVTTAVAWYQQKPGKSPKLLIYSASYRYTGVPSRFSGSGSGTDFTFTISSVQPEDLATYYCQQHYSAPWTFGGGTNLEIK

SEQ ID NO: 326 - protein, 116 aa, unidentified
QVQLVQSGAEVVKPGASVKVSCKTSGYTFTEYTIHWVRQAPGQSLEWIGGINPNNGGTVYNQKFQGKVTLTVDKSTSTAYMELSSLRSEDTAVYYCARVRRGMDYWGQGTSVTVSS

SEQ ID NO: 327 - protein, 107 aa, unidentified
DIQMTQSPSSLSASVGDRVTITCRASQDVTTAVAWYQQKPGKAPKLLIYSASYRYTGVPSRFSGSGSGTDFTLTISSVQPEDLATYYCQQHYSAPWTFGGGTNLEIK

SEQ ID NO: 328 - protein, 117 aa, unidentified
EVQLVESGGGLVQPGGSLRLSCAASGIDLRTNSMSWVRQAPGKGLEWVGIIGSSGYMDYASWAKGRFTISRDNSKNTLYLQMNSLRAEDTAVYYCARHGDSSSFALWGQGTLVTVSS

SEQ ID NO: 329 - protein, 110 aa, unidentified
DIQMTQSPSSLSASVGDRVTITCRASESVYKNNRLSWYQQKPGKAPKLLIYEASKVASGVPSRFSGSGSGTDFTLTISSLQPEDFATYYCAGAYRGNIYPFGQGTKVEIK

SEQ ID NO: 330 - protein, 116 aa, unidentified
EVQLVESGGGLVQPGGSLRLSCAASGFSLNSNAVGWVRQAPGKGLEYIGFINFSGITYYANWAKGRFTISRDNSKNTLYLQMNSLRAEDTAVYYCARGSAGSFDLWGQGTLVTVSS

SEQ ID NO: 331 - protein, 110 aa, unidentified
DIQMTQSPSSLSASVGDRVTITCQASESVYKNNYLAWYQQKPGKAPKLLIYRASNLASGVPSRFSGSGSGTDFTLTISSLQPEDFATYYCQGGYSSGMYPFGQGTKVEIK

SEQ ID NO: 332 - protein, 118 aa, unidentified
EVQLVESGGGLVKPGGSLKLSCAASGFTFSDYGMHWVRQAPEKGLEWVAFISSGSSIIYYADIVKGRSTISRDNAKNTLFLQMTSLRSEDTAMYYCTRGNKRGFFDYWGQGTILTVSS

SEQ ID NO: 333 - protein, 108 aa, unidentified
QIVLTQSPALMSASPGEKVTMTCNASSSVSYMYWYQRKPRSSPKPWIYLTSNLASGVPVRFSGSGSGTSYSLTISSMEAEDAATYYCQQWRSNPPMLTFGSGTKLEVK

SEQ ID NO: 334 - protein, 118 aa, unidentified
EVQLVESGGGLVQPGGSLRLSCAASGFTFSDYGMHWVRQAPGKGLEWVAFISSGSSIIYYADIVKGRSTISRDNAKNTLYLQMNSLRAEDTAVYYCTRGNKRGFFDYWGQGTLVTVSS

SEQ ID NO: 335 - protein, 108 aa, unidentified
EIVLTQSPATLSLSPGERATLSCRASSSVPYMYWYQQKPGQAPRLLIYLTSNLASGIPARFSGSGSGTDFTLTISSLEPEDFAVYYCQQWRAYPPMLTFGGGTKVEIK

SEQ ID NO: 336 - protein, 108 aa, unidentified
EIVLTQSPATLSLSPGERATLSCRASPGVPPLAWYQQKPGQAPRLLIYLASSRPSGIPARFSGSGSGTDFTLTISSLEPEDFAVYYCQQWRSNPPMLTFGGGTKVEIK

SEQ ID NO: 337 - protein, 122 aa, unidentified
EVQLVESGGGLVKPGGSLKLSCAASGFTFSDYGMHWVRQAPEKGLEWVAYISSGSTTIYYADTVKGRFTISRDNGKNTLFLQMTSLRSEDTAMYYCARISTVVAKRYAMDYWGQGTSVTVSS

SEQ ID NO: 338 - protein, 107 aa, unidentified
DIQMTQTASSLSASLGDRVTISCRASQDISNYLNWYQQKPDGTVKLLIYYTSRLHSGVPSRFSGSGSGTDYSLTISNLEQEDIATYFCQQINALPLTFGAGTKLELK

SEQ ID NO: 339 - protein, 122 aa, unidentified
EVQLVESGGGLVQPGGSLRLSCAASGFTFSDYGMHWVRQAPGKGLEWVSYISSGSTTIYYADTVKGRFTISRDNAKNSLYLQMNSLRAEDTAVYYCARISTVVAKRYAMDYWGQGTLVTVSS

SEQ ID NO: 340 - protein, 107 aa, unidentified
DIQMTQSPSSLSASVGDRVTITCRASQDISNYLNWYQQKPGKAPKLLIYYTSRLHSGVPSRFSGSGSGTDFTLTISSLQPEDIATYYCQQINALPFTFGQGTKLEIK

SEQ ID NO: 341 - protein, 125 aa, unidentified
EVQLVESGGGLVQPGGSLRLSCAVSGFTFSSYAMSWVRQAPGKGLEWVSSITGGGGGIYYADSVKGRFTISRDNSKNTVYLQMNSLRAEDTAVYYCAKDRISITIRPRYFGLDFWGQGTTVTVSS

SEQ ID NO: 342 - protein, 112 aa, unidentified
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQSFKAPYTFGQGTKLEIK

SEQ ID NO: 343 - protein, 112 aa, unidentified
DIVMTQSPLSLPVTPGEPASISCRSSRNVIYGNGYNYLDWYLQKSGQSPQLLIYLGNNVAAGVPDRFSGSGSGTDFTLKISRVEAEDVGFYYCMQSLQAPYTFGQGTKLEIK

SEQ ID NO: 344 - protein, 112 aa, unidentified
DIVMTQSPLSLPVTPGEPASISCRSSQNVVYGNGYNYLDWYLQKSGQSPQLLIYLGTNVAAGVPDRFSGSGSGTDFTLKISRVEAEDVGFYYCMQSLQAPYTFGQGTKLEIK

SEQ ID NO: 345 - protein, 112 aa, unidentified
DIVMTQSPLSLPVTPGEPASISCRSSQNVVYGNGYNYLDWYLQKSGQSPQLLIYLGNNVAAGVPDRFSGSGSGTDFTLKISRVEAEDVGFYYCMQSLKAPYTFGQGTKLEIK

SEQ ID NO: 346 - protein, 112 aa, unidentified
DIVMTQSPLSLPVTPGEPASISCRSSHNLLYSNGYNYLDWYLQKPGQSPQLLIYLGSNRAYGVPDRFSGSGSGTDFTLKISRVEAEDVGVYHCMQALQSPYTFGQGTKVDIK

SEQ ID NO: 347 - protein, 112 aa, unidentified
DIVMTQSPLSLPVTAGAPASISCRSSQSLLYSNGYNYLDWYMQKPGQSPQLLIYLGSNRASGVPDRFSGSGSGTDFTLKISRVEAEDVGVYYCMQALETPYAFGQGTKVEIK

SEQ ID NO: 348 - protein, 120 aa, unidentified
QVQLQQSGPELVKPGASVKISCTASGSTLTDDYINWVKQRPGRGLEWVGWIFPGNGNSYYNEKFKDKATLIVDKSSSTAYMLLSSLTSEDSAVYFCARGLVRYRALFDYWGQGTTLTVSS

SEQ ID NO: 349 - protein, 106 aa, unidentified
QIVLSQSPAILSASPGEKVTMTCRASSSINYMHWYQQKPGSSPKPWIYAASNLASGVPARFSGSGSGTSFSLTISRVEAEDAATYYCQQWSSYPITFGSGTKLEIK

SEQ ID NO: 350 - protein, 120 aa, unidentified
QVQLVQSGAEVKKPGASVKVSCKASGSTLTDDYINWVRQAPGQRLEWVGWIFPGNGNSYYNEKFKDRATLTVDKSASTAYMELSSLRSEDTAVYFCARGLVRYRALFDYWGQGTLVTVSS

SEQ ID NO: 351 - protein, 106 aa, unidentified
DIQMTQSPSSLSASVGDRVTITCRASSSINYMHWYQQKPGKAPKPWIYAASNLASGVPSRFSGSGSGTDFTLTISSLQPEDFATYYCQQWSSYPITFGQGTKVEIK

SEQ ID NO: 352 - protein, 119 aa, unidentified
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGLTTVRGVLYWGQGTLVTVSS

SEQ ID NO: 353 - protein, 107 aa, unidentified
EIVMTQSPSSLSASLGDRVTITCRASQNIGSRLAWYQQKPGKAPKLLIYKASSLESGVPSRFSGSGSGTEFTLTISSLQPDDFATYYCQQYNSYSWTFGQGTKLEIK

SEQ ID NO: 354 - protein, 119 aa, unidentified
EVQLVESGGGLVQPGGSLRLSCAASGFTFSTYGMSWVRQAPGKGLVXVXTINSNGGSTSYPDSVKGRFTISRDNAKNTLYLQMNSLRAEDTAVYYCARFFRFRNAMDYWGQGTLVTVSS

SEQ ID NO: 355 - protein, 112 aa, unidentified
DIVMTQTPLSLSVTPGQPASISCRSSQSIVHSNGNTYLEWYLQKPGQSPQLLIYKVTNRFSGVPDRFSGSGSGTDFTLKISRVEAEDVGVYYCFQGSHVPYTFGQGTKLEIK

SEQ ID NO: 356 - protein, 116 aa, unidentified
EVQLVQSGAEVKKPGATVKISCKVSGFNIKDTYMHWVQQAPGKGLEWMGLIDPTNGYTIYAEKFQGRVTITADTSTDTAYMELSSLRSEDTAVYYCVSRRPWFAYWGQGTLVTVSS

SEQ ID NO: 357 - protein, 116 aa, unidentified
QVQLQQSGAELVKPGASVKLSCTASGFNIKDTYMHWVKQRPEQGLEWVGRIDPTNGYTIYASKFQGKATITADTSSNTAYMQLSSLTSEDTAVYHCVSRRPWFAYWGQGTTLTVSS

SEQ ID NO: 358 - protein, 120 aa, unidentified
EVQLVESGGGLVKPGGSLKLSCAASGFTFSSYAMSWVRQTPEKRLEWVAGIRSGGSYTYYPDTVKGRFTISRDNARNTLYLQMNSLRSEDTAIYYCARGDKLRPYHFDYWGQGTTLTVSS

SEQ ID NO: 359 - protein, 107 aa, unidentified
DIVMTQSHKFMSTSVGDKVSITCKASQDVTTAVAWYQQKPGQSPKLLIYSASYRYTGVPDRFAGSGSGTDFTVTISTVQAEDLAVYYCQQHYSDPYTFGGGTKLEIK

SEQ ID NO: 360 - protein, 120 aa, unidentified
EVQLVESGGGLVKPGGSLKLSCAASGFTFSNYAMSWVRQTPEKRLEWVAGIRSGGSYTYYPDTVKGRFTISRDNARNTLYLQMSSLRSEDTAIYYCARGDKLRPYHFDYWGQGTTLTVSS

SEQ ID NO: 361 - protein, 107 aa, unidentified
DIVMTQSHKFMSTSVGDRVSITCKASQDVSTAVVWYQQKPGQSPKLLIYSASYRYTGVPDRFTGSGSGTDFTFTIITVQAEDLAVYYCQQHYSAPYTFGGGTQLEIK

SEQ ID NO: 362 - protein, 115 aa, unidentified
EVKLVESGGGLVKPGGSLKLSCAASGFTFSSYAMSWVRQTPEKRLEWVASISSGDSTYYLDSVKGRFTISRDNARNILYLQVSSLRSEDTAMYYCERSGGSAPYWGQGTLVTVSA

SEQ ID NO: 363 - protein, 106 aa, unidentified
QIVLTQSPAIMSASPGDMVTISCSASSSVNYMYWYQQKPGSSPKPWIYRTSNLASGVPARFSGSGSGTSYSLTISSMEAEDAATYYCQQYHSFPLTFGAGTKLELK

SEQ ID NO: 364 - protein, 115 aa, unidentified
EVKLVESGGGLVKPGGSLKLSCAASGFTFSSYAMSWVRQTPEKRLEWVASISSGDSTYYLDSVKGRFTISRDNAMNILYLQMSSLRSEDTAVYYCERSGGSAPYWGQGTLVSVSA

SEQ ID NO: 365 - protein, 106 aa, unidentified
QIVLTQSPAIMSASPGEMVTISCSASSSVSYMYWYQQKPGSSPKPWIYRTSNLASGVPARFSGSGSGTSYSLTISSMEAEDAATYYCQQYHSFPLTFGAGTKLELK

SEQ ID NO: 366 - protein, 452 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK